(19) United States
(12) Patent Application Publication
(10) Pub. No.: US 2022/0154242 A1
(43) Pub. Date: May 19, 2022

(54) METHOD FOR MODIFYING SUBSTRATE SPECIFICITY OF GLUCOSE DEHYDROGENASE AND AGENT FOR MODIFYING SUBSTRATE SPECIFICITY OF GLUCOSE DEHYDROGENASE

(71) Applicant: KIKKOMAN CORPORATION, Noda-shi, Chiba (JP)

(72) Inventors: Yosuke MASAKARI, Noda-shi, Chiba (JP); Miho TOYOOKA, Noda-shi, Chiba (JP)

(73) Assignee: KIKKOMAN CORPORATION, Noda-shi, Chiba (JP)

(21) Appl. No.: 17/439,568

(22) PCT Filed: Mar. 26, 2020
(86) PCT No.: PCT/JP2020/013544

§ 371 Date: Sep. 15, 2021

(30) Foreign Application Priority Data
Mar. 26, 2019 (JP) 2019-058223
Oct. 3, 2019 (JP) 2019-182592

(51) Int. Cl. C12Q 1/00; C12Q 1/32; C12N 9/04

(52) U.S. Cl. CPC C12Q 1/006; C12Y 101/05; C12N 9/0006; C12Q 1/32

(57) ABSTRACT

An object to modify the substrate specificity of glucose dehydrogenase is provided. Also provided are a method of modifying the substrate specificity of glucose dehydrogenase by using a glucose dehydrogenase substrate specificity modifier, the modifier being a glucose analog and a low molecular weight compound, as well as a glucose dehydrogenase substrate specificity modifier.

### TECHNICAL FIELD

The present disclosure relates to a method of modifying the substrate specificity of glucose dehydrogenase and a glucose dehydrogenase substrate specificity modifier.

### BACKGROUND ART

Glucose measurement is used, for example, for monitoring blood glucose of diabetic patients. Quantification of glucose is usually carried out by using glucose oxidase (hereinafter, also referred to as GOD), glucose dehydrogenase (hereinafter, also referred to as GDH), or the like.

Glucose oxidase is an oxidoreductase that catalyzes the oxidation reaction of β-D-glucose to D-glucono-1,5-lactone (gluconolactone). Glucose oxidase uses oxygen as an electron acceptor and flavin adenine dinucleotide (FAD) as a cofactor.

Glucose dehydrogenase, which is classified as an oxidoreductase, catalyzes the reaction that uses glucose and an electron acceptor as substrates to generate gluconolactone and a reduced acceptor. Examples of glucose dehydrogenase include nicotinamide dinucleotide-dependent GDH, nicotinamide dinucleotide phosphate-dependent GDH, pyrroloquinoline quinone (PQQ)-dependent GDH, and FAD-dependent GDH (flavin binding GDH).

When an enzyme using glucose as a substrate is used to measure blood glucose, the substrate specificity of the enzyme can be problematic. In particular, the reactivity of the enzyme to maltose caused problems in some cases. For example, wild-type PQQ-GDH is not specific to glucose and reacts with other sugars such as maltose, galactose, and xylose. Non Patent Literature 1 discloses a problem in that, with regard to glucose measurements using a PQQ-GDH system, due to the presence of maltose or other sugars in the system, a glucose level higher than the actual level is falsely obtained, which leads to hypoglycemia. Accordingly, for glucose measurements using enzymes, there has been a need for a measurement reagent having a high specificity for glucose and a low reactivity to other sugars such as maltose.

To enhance the substrate specificity for glucose or reduce the reactivity to maltose, some approaches for searching for a glucose dehydrogenase (GDH) with an excellent substrate specificity were attempted, and various FAD-dependent GDHs were found (see Non Patent Literatures 2 and 3). Another approach has also been attempted, wherein the gene of an isolated GDH is obtained and the gene is genetically engineered, to modify the substrate specificity of the GDH.

On the other hand, apart from the specificity for glucose, the stability of the enzyme can be problematic in some cases. Patent Literature 1 (JP Patent Publication (Kokai) No. 2015-139376 A) discloses a method for improving the stability of glucose dehydrogenase by adding smectite, which is a mineral.

It is an object of Patent Literature 2 (International Publication No. WO 2005/054840) to provide a method for measuring a blood component by measuring the hematocrit value of the blood with high precision and high reliability, wherein the amount of the blood component can be sufficiently and accurately corrected; as well as a sensor and an apparatus used therefor. In this connection, Patent Literature 2 discloses an apparatus comprising glucose dehydrogenase. Paragraph 0027 lists sugar alcohols as enzyme-stabilizing agents and describes maltitol as a preferred agent. In the Examples, maltitol is used.

It is an object of Patent Literature 3 (JP Patent Publication (Kokai) No. 2005-114359 A) to provide a method for measuring components without requiring a complicated correction process and a sensor used therefor. In this connection, Patent Literature 3 discloses a method for measuring glucose in blood and a sensor used therefor. Paragraph 0016 therein lists sugar alcohols as enzyme-stabilizing agents and describes maltitol as a preferred agent. In the Examples, maltitol is used.

Patent Literature 4 (International Publication No. WO 2001/025776) discloses a glucose sensor using PQQ-GDH. This patent states, on page 8, that a stabilizer may be added to the reaction layer of the biosensor and discloses sugar as the stabilizer. Furthermore, examples of such sugar are listed, including glucose and maltose. In the Examples, there is no description of using sugar as the stabilizer.

Patent Literature 5 (JP Patent Publication (Kokai) No. 2009-195250 A) discloses a method for improving the stability of a composition comprising a soluble glucose dehydrogenase. This patent also discloses a composition comprising a recombinant FAD-GDH from Aspergillus oryzae or Aspergillus terreus and trehalose.

Patent Literature 6 (JP Patent Publication (Kokai) No. 2014-018096 A) discloses a method for improving the stability of a composition comprising a flavin binding glucose dehydrogenase.

### CITATION LIST

### Patent Literature

### Patent Literature 1

JP Patent Publication (Kokai) No. 2015-139376 A (JP Patent No. 6402887)

### Patent Literature 2

International Publication No. WO 2005/054840

### Patent Literature 3

JP Patent Publication (Kokai) No. 2005-114359 A

### Patent Literature 4

International Publication No. WO 2001/025776

### Patent Literature 5

JP Patent Publication (Kokai) No. 2009-195250 A (JP Patent No. 5176045)

### Patent Literature 6

JP Patent Publication (Kokai) No. 2014-018096 A (JP Patent No. 6101011)

### Non Patent Literature

### Non Patent Literature 1

Frias, et al., Diabetes Care, Vol. 33, No. 4, April 2010, pp. 728-729

### Non Patent Literature 2

Tsujimura S, et al., Biosci Biotechnol Biochem., Vol. 70, 2006, pp. 654-659

### Non Patent Literature 3

Satake R, et al., J Biosci Bioeng., Vol. 120, 2015, pp. 498-503

### SUMMARY OF INVENTION

### Technical Problem

An object of the present disclosure is to provide a method of modifying the substrate specificity of an FAD-dependent glucose dehydrogenase by using a low molecular weight compound that is a glucose analog; and a glucose dehydrogenase substrate specificity modifier (substrate specificity modifying agent) that is a glucose analog and a low molecular weight compound.

### Solution to Problem

In view of the above described problems, the present inventors have conducted intensive studies on a method of modifying the reaction of GDH with non-glucose sugars such as maltose. For this purpose, the present inventors used a method different from an approach based on searching for a novel GDH enzyme or an approach that involves introducing mutation into an existing GDH to modify the substrate specificity thereof. As a result, the present inventors have found that a low molecular weight compound that is a certain glucose analog surprisingly modifies the reactivity of an FAD-dependent glucose dehydrogenase to non-glucose sugars (e.g., maltose) thereby completing (accomplishing) the present invention.

Thus, the present disclosure encompasses the following embodiments.

[1] A method of modifying the substrate specificity of glucose dehydrogenase by using a glucose dehydrogenase substrate specificity modifier, said modifier being a glucose analog and a low molecular weight compound, wherein the method comprises allowing the glucose dehydrogenase substrate specificity modifier and the glucose dehydrogenase to coexist when glucose measurement is carried out using an FAD-dependent glucose dehydrogenase (FAD-GDH),
wherein the ratio (Mal/Glu) of the reactivity of the glucose dehydrogenase to maltose (Mal) relative to the reactivity thereof to glucose (Glu) in the presence of the modifier is altered, compared to the ratio (Mal/Glu) of the reactivity of the glucose dehydrogenase to maltose (Mal) relative to the reactivity thereof to glucose (Glu) in the absence of the modifier.
[2] The method according to embodiment 1, wherein the glucose dehydrogenase substrate specificity modifier comprises one or more compounds selected from the group consisting of sorbitol, D-iditol, L-iditol, D-glucal, ribitol, L-gulose, trehalose, D-mannitol, xylitol, and glycerol.
[3] The method according to embodiment 1 or 2, wherein the glucose dehydrogenase is a glucose dehydrogenase from a microorganism of the genus Mucor or the genus Aspergillus.
[4] The method according to any of embodiments 1 to 3, wherein the glucose dehydrogenase is immobilized onto a solid-phase surface.
[5] The method according to any of embodiments 1 to 3, wherein the glucose dehydrogenase is not immobilized onto a solid-phase surface.
[6] A glucose dehydrogenase substrate specificity modifier, said modifier being a glucose analog and a low molecular weight compound, wherein the ratio (Mal/Glu) of the reactivity of an FAD-dependent glucose dehydrogenase (FAD-GDH) to maltose (Mal) relative to the reactivity thereof to glucose (Glu) in the presence of the modifier is altered, compared to the ratio (Mal/Glu) of the reactivity of the glucose dehydrogenase to maltose (Mal) relative to the reactivity thereof to glucose (Glu) in the absence of the modifier.
[7] The modifier according to embodiment 6, wherein the glucose dehydrogenase substrate specificity modifier comprises one or more compounds selected from the group consisting of sorbitol, D-iditol, L-iditol, D-glucal, ribitol, L-gulose, trehalose, D-mannitol, xylitol, and glycerol.
[8] The modifier according to embodiment 6, wherein the glucose dehydrogenase substrate specificity modifier comprises one or more compounds selected from the group consisting of sorbitol, D-iditol, L-iditol, D-glucal, ribitol, L-gulose, D-mannitol, and glycerol.
[9] The modifier according to any of embodiments 6 to 8, wherein the glucose dehydrogenase is a glucose dehydrogenase from a microorganism of the genus Mucor or the genus Aspergillus.
[10] A system for glucose measurement, comprising the glucose dehydrogenase substrate specificity modifier recited in any of embodiments 6 to 9 and a glucose dehydrogenase immobilized onto a solid-phase surface.
[11] A system for glucose measurement, comprising the glucose dehydrogenase substrate specificity modifier recited in any of embodiments 6 to 9 and a glucose dehydrogenase that is not immobilized onto a solid-phase surface.
[12] A composition for glucose measurement or a reagent for glucose measurement, wherein the composition or the reagent comprises the glucose dehydrogenase substrate specificity modifier recited in any of embodiments 6 to 9 and glucose dehydrogenase.
[13] The composition for glucose measurement or the reagent for glucose measurement according to embodiment 12, wherein the composition or the reagent comprises the glucose dehydrogenase substrate specificity modifier and the glucose dehydrogenase as separate reagents.
[14] The composition for glucose measurement or the reagent for glucose measurement according to embodiment 12, wherein the composition or the reagent comprises the glucose dehydrogenase substrate specificity modifier and the glucose dehydrogenase in a single reagent.
[15] A glucose measurement method that uses the modifier recited in any of embodiments 6 to 9, the system recited in embodiment 10 or 11, or the composition or reagent recited in any of embodiments 12 to 14.
[16] A screening method for a glucose dehydrogenase substrate specificity modifier, comprising the steps of:
i) providing a glucose dehydrogenase;
ii) determining the ratio (Mal/Glu) of the reactivity of the glucose dehydrogenase to maltose (Mal) relative to the reactivity thereof to glucose (Glu);
iii) contacting the glucose dehydrogenase described above in i) with a candidate substance that is a glucose analog and a low molecular weight compound, and then determining the ratio (Mal/Glu) of the reactivity of the glucose dehydrogenase to maltose (Mal) relative to the reactivity thereof to glucose (Glu) in the presence of the candidate substance;
iv) comparing the ratio (Mal/Glu) obtained in ii) with the ratio (Mal/Glu) obtained in iii) in the presence of the candidate substance; and
v) identifying the candidate substance as a glucose dehydrogenase substrate specificity modifier if the ratio (Mal/Glu) obtained in iii) in the presence of the candidate substance is altered compared to the ratio (Mal/Glu) obtained in ii).
[17] A method for producing a reagent for glucose measurement or a composition for glucose measurement, comprising the step of incorporating a glucose dehydrogenase substrate specificity modifier identified by the method recited in embodiment 16 into the reagent for glucose measurement or the composition for glucose measurement.
[18] A reagent for glucose measurement, comprising a glucose dehydrogenase substrate specificity modifier identified by the method recited in embodiment 16 and glucose dehydrogenase.
[19] An electrode comprising a glucose dehydrogenase substrate specificity modifier and an FAD-dependent glucose dehydrogenase (FAD-GDH), wherein the glucose dehydrogenase substrate specificity modifier comprises one or more compounds selected from the group consisting of sorbitol, D-iditol, L-iditol, D-glucal, ribitol, L-gulose, trehalose, D-mannitol, xylitol, and glycerol.
[20] An electrical cell comprising a glucose dehydrogenase substrate specificity modifier and an FAD-dependent glucose dehydrogenase (FAD-GDH), wherein the glucose dehydrogenase substrate specificity modifier comprises one or more compounds selected from the group consisting of sorbitol, D-iditol, L-iditol, D-glucal, ribitol, L-gulose, trehalose, D-mannitol, xylitol, and glycerol.

The present specification encompasses the contents disclosed in JP Patent Application No. 2019-058223 and JP Patent Application No. 2019-182592 based on which the present application claims priority.

### Advantageous Effects of Invention

According to the present disclosure, the substrate specificity of glucose dehydrogenase can be modified.

### DESCRIPTION OF EMBODIMENTS

In one embodiment, the present disclosure provides a glucose dehydrogenase substrate specificity modifier (substrate specificity modifying agent). The substrate specificity modifier of the present disclosure may be a glucose analog (analogous substance). In the present specification, a “glucose analog” refers to a sugar compound or a sugar alcohol compound that has a chemical structure similar to that of glucose but is not recognized as a substrate by glucose dehydrogenase. The glucose analog may be a low molecular weight compound. As used herein, a “low molecular weight compound” refers to a compound with a molecular weight (g/mol) of about 90 to about 380, for example about 150 to about 380. The expression “not recognized as a substrate by glucose dehydrogenase” means not being recognized as a substrate at all or substantially not being recognized as a substrate by glucose dehydrogenase. “Substantially not being recognized as a substrate” means specifically that when the activity (reactivity) of glucose dehydrogenase to glucose, which serves as the substrate, is considered as 100%, the activity thereof to a glucose analog is 1% or less, 0.5% or less, 0.1% or less, 0.05% or less, 0.01% or less, 0.005% or less, or 0.001% or less, for example 0.0005% or less.

In one embodiment, the glucose dehydrogenase substrate specificity modifier of the present disclosure comprises one or more compounds selected from the group consisting of L-gulose, D-iditol, L-iditol, sorbitol, ribitol (CAS No. 488-81-3, also referred to as adonitol), trehalose, and D-glucal, as well as D-mannitol, xylitol, and glycerol. In another embodiment, the substrate specificity modifier of the present disclosure is L-gulose, D-iditol, L-iditol, sorbitol, ribitol, trehalose, D-glucal, D-mannitol, xylitol, glycerol, or a combination of one or more thereof.

When the substrate specificity modifier of the present disclosure comprises L-gulose (CAS No. 6027-89-0), the L-gulose may be provided as a mixture of the L-form and D-form thereof, for example a racemate, or as the L-form thereof. In the present specification, “the L-form of an optically active compound” means that the L-form thereof exists at an optical purity of 90 to 100% ee, for example 95 to 100% ee, for example 97 to 100% ee. For example, the L-gulose may be one with an optical purity of 90 to 100% ee, for example 95 to 100% ee, for example 97 to 100% ee.

When the substrate specificity modifier of the present disclosure comprises D-iditol (CAS No. 25878-23-3), the D-iditol is preferably provided as the D-form thereof. In the present specification, “the D-form of an optically active compound” means that the D-form thereof exists at an optical purity of for example 90 to 100% ee, for example 95 to 100% ee, for example 97 to 100% ee. For example, the D-iditol may be one with an optical purity of 90 to 100% ee, for example 95 to 100% ee, for example 97 to 100% ee.

When the substrate specificity modifier of the present disclosure comprises L-iditol (CAS No. 488-45-9), the L-iditol is preferably provided as the L-form thereof. For example, the L-iditol may be one with an optical purity of 90 to 100% ee, for example 95 to 100% ee, for example 97 to 100% ee.

When the substrate specificity modifier of the present disclosure comprises D-glucal (CAS No. 13265-84-4), the D-glucal is preferably provided as the D-form thereof. For example, the D-glucal may be one with an optical purity of 90 to 100% ee, for example 95 to 100% ee, for example 97 to 100% ee.

When the substrate specificity modifier of the present disclosure comprises D-mannitol (CAS No. 69-65-8), the D-mannitol is preferably provided as the D-form thereof. For example, the D-mannitol may be one with an optical purity of 90 to 100% ee, for example 95 to 100% ee, for example 97 to 100% ee.

In one embodiment, the glucose dehydrogenase substrate specificity modifier of the present disclosure does not comprise a smectite.

The glucose dehydrogenase substrate specificity modifier of the present disclosure may be incorporated into a reagent for glucose measurement or a composition for glucose measurement so that the final concentration thereof in a measurement solution at the time of glucose measurement becomes 0.1 mM to 1000 mM, for example 0.1 mM to 500 mM, 0.1 mM to 300 mM, 0.1 mM to 100 mM, 0.1 mM to 80 mM, 0.1 mM to 70 mM, 0.1 mM to 60 mM, 0.1 mM to 50 mM, 0.2 mM to 20 mM, or 0.25 mM to 10 mM, for example 5 mM. In one embodiment, an aqueous solution containing the glucose dehydrogenase substrate specificity modifier of the present disclosure and glucose dehydrogenase can be applied onto an electrode and dried. In such a case, the glucose dehydrogenase substrate specificity modifier is believed to be concentrated during drying and consequently have a higher concentration than the final concentration before drying. Such a glucose dehydrogenase substrate specificity modifier at a higher concentration after drying and an electrode onto which the modifier was applied are also encompassed by the present disclosure.

In one embodiment, the present disclosure provides a composition for glucose measurement or a reagent for glucose measurement, comprising a glucose dehydrogenase substrate specificity modifier and glucose dehydrogenase. In one embodiment, the reagent for glucose measurement may be in a solution form or in a dried form. In one embodiment, the glucose dehydrogenase substrate specificity modifier and the glucose dehydrogenase in the reagent for glucose measurement may be included as separate reagents. In another embodiment, the glucose dehydrogenase substrate specificity modifier and the glucose dehydrogenase in the reagent for glucose measurement may be included (comprised) in a single reagent. In a further embodiment, the reagent for glucose measurement does not comprise the glucose dehydrogenase, and in this case, the glucose dehydrogenase may be added to the measurement solution at the time of glucose measurement. In another embodiment, the reagent for glucose measurement does not comprise the glucose dehydrogenase substrate specificity modifier, and in this case, the glucose dehydrogenase substrate specificity modifier may be added to the measurement solution at the time of glucose measurement.

In one embodiment, the present disclosure provides a glucose measurement method using the substrate specificity modifier and glucose dehydrogenase. The glucose measurement method may be a method of measuring glucose concentration. The glucose measurement method may be a method of quantifying glucose concentration. The sample to be measured may be a sample comprising glucose. The sample to be measured may be a sample comprising maltose. The glucose dehydrogenase substrate specificity modifier may be included in advance in the reagent for glucose measurement or may be added to the measurement solution at the time of glucose measurement.

In the glucose measurement method of the present disclosure, the glucose dehydrogenase substrate specificity modifier may be included, at the time of glucose measurement, in the measurement solution at a final concentration of 0.1 mM to 1000 mM, for example 0.1 mM to 500 mM, 0.1 mM to 300 mM, 0.1 mM to 100 mM, 0.1 mM to 80 mM, 0.1 mM to 70 mM, 0.1 mM to 60 mM, 0.1 mM to 50 mM, 0.2 mM to 20 mM, or 0.25 mM to 10 mM, for example 5 mM.

In one embodiment, in the glucose measurement method of the present disclosure, the glucose dehydrogenase substrate specificity modifier may be included in the glucose measurement solution. In this case, the measurement may be initiated by adding glucose dehydrogenase and a sample to the glucose measurement solution. Alternatively, the glucose measurement may be carried out by immobilizing glucose dehydrogenase onto an electrode and then contacting the glucose measurement solution and a sample with the electrode.

In another embodiment, the glucose dehydrogenase substrate specificity modifier of the present disclosure may be included in the reagent for glucose measurement in advance. This measurement reagent may or may not comprise glucose dehydrogenase. When the measurement reagent comprises a glucose dehydrogenase, the glucose measurement can be initiated by adding a sample to the measurement reagent. When the measurement reagent does not comprise glucose dehydrogenase, the glucose measurement can be initiated by adding a sample and glucose dehydrogenase (in any order) to the measurement reagent. Alternatively, the glucose measurement may be carried out by immobilizing glucose dehydrogenase onto an electrode and then contacting the measurement reagent and a sample with the electrode.

In the present specification, unless stated otherwise, glucose dehydrogenase refers to FAD-dependent glucose dehydrogenase (FAD-GDH). As the glucose dehydrogenase, a known FAD-GDH can be used. Suitable example of microorganisms from which known FAD-GDHs are derived include microorganisms classified in the subbranch Mucor, preferably the class Mucor, more preferably the order Mucor and further preferably the family Mucor. Specific examples thereof include FAD-GDHs derived from the genus Mucor, the genus Absidia, the genus Actinomucor and the genus Circinella.

Specific examples of preferable microorganism classified in the genus Mucor include Mucor prainii, Mucor javanicus, Mucor circinelloides f. circinelloides, Mucor guilliermondii, Mucor hiemalis f. silvaticus, Mucor subtilissimus, Mucor dimorphosporus and the like. More specific examples thereof include Mucor prainii, Mucor javanicus, Mucor circinelloides f. circinelloides, Mucor guilliermondii NBRC9403, Mucor hiemalis, Mucor hiemalis f. silvaticus NBRC6754, Mucor subtilissimus NBRC6338, Mucor RD056860, Mucor dimorphosporus NBRC5395 and the like. Specific examples of preferable microorganisms classified in the genus Absidia include Absidia cylindrospora and Absidia hyalospora. Specific examples of preferable microorganism classified in the genus Actinomucor include Actinomucor elegans. Specific examples of preferable microorganism classified in the genus Circinella include Circinella minor, Circinella mucoroides, Circinella muscae, Circinella rigida, Circinella simplex, and Circinella umbellata. More specific examples thereof include Circinella minor NBRC6448, Circinella mucoroides NBRC4453, Circinella muscae NBRC6410, Circinella rigida NBRC6411, Circinella simplex NBRC6412, Circinella umbellata NBRC4452, Circinella umbellata NBRC5842, Circinella RD055423 and Circinella RD055422. Incidentally, NBRC strains and RD strains are strains deposited in the NBRC (the Biotechnology Center of the National Institute of Technology and Evaluation). These FAD-GDHs encompass mutants thereof.

Examples of other known FAD-GDHs include FAD-GDHs from a microorganism of the genus Aspergillus, such as a GDH from Aspergillus oryzae, a GDH from Aspergillus awamori, a GDH from Aspergillus aureus, a GDH from Aspergillus niger, a GDH from Aspergillus foetidus, a GDH from Aspergillus iizukae, a GDH from Aspergillus versicolor, a GDH from Aspergillus phoenicis, a GDH from Aspergillus bisporus, a GDH from Aspergillus brunneo-uniseriatus, a GDH from Aspergillus carneus, a GDH from Aspergillus malignus, and a GDH from Aspergillus terreus. These FAD-GDHs encompass mutants thereof.

Examples of other known FAD-GDHs include FAD-GDHs from a microorganism of the genus Glomerella, such as a GDH from Glomerella fructigena, for example a GDH from Glomerella fructigena NBRC5951 strain and a GDH from Glomerella cingulata, for example a GDH from Glomerella cingulata NBRC107000 strain; FAD-GDHs from a microorganism of the genus Colletotrichum, such as a GDH from Colletotrichum gloeosporioides, a GDH from Colletotrichum chlorophyti, and a GDH from Colletotrichum orbiculare; and FAD-GDHs from a microorganism of the genus Botryosphaeria, such as a GDH from Botryosphaeria parva. Further examples thereof include FAD-GDHs from a microorganism of the genus Burkholderia, such as a GDH from Burkholderia cepacia, which is a membrane protein, and a GDH from Burkholderia cepacia KS1 strain. See, for example, JP Patent Publication (Kokai) No. 2019-000020 A (JP Patent No. 6453385), International Publication No. WO2017/002896, International Publication No. WO2002/036779 (JP Patent No. 4107386). These FAD-GDHs encompass mutants thereof.

### (Obtaining a Gene Encoding an FAD-GDH)

A gene encoding an FAD-GDH can be obtained using genetic engineering techniques. In order to obtain a gene encoding an FAD-GDH (hereinafter referred to as FAD-GDH gene), gene cloning methods commonly used may be employed. For example, chromosomal DNA or mRNA can be extracted from known microbial cells and various cells having FAD-GDH producing capability in accordance with routine methods, for example, methods described in Current Protocols in Molecular Biology (WILEY Interscience, 1989). Further, cDNA can be synthesized by using mRNA as the template. Using chromosomal DNA or cDNA thus obtained, a library of chromosomal DNA or cDNA can be prepared.

Next, an appropriate probe DNA can be synthesized based on the amino acid sequence information of a known FAD-GDH and an FAD-GDH gene having high substrate specificity can be screened from the library of chromosomal DNA or cDNA by using the probe DNA. Alternatively, appropriate primer DNAs can be prepared based on the amino acid sequence above and subjected to an appropriate polymerase chain reaction (PCR method) such as 5′RACE method and 3′RACE method to amplify the DNA containing the gene fragment of interest encoding the FAD-GDH having high substrate specificity and then these DNA fragments can be linked (ligated) to obtain DNA comprising a full-length FAD-GDH gene of interest.

A method can be adopted in which a mutation is introduced into the gene encoding the obtained FAD-GDH and selection of the FAD-GDHs expressed by various mutant genes is carried out based on enzymological properties as the index (indicator).

Mutation for the starting FAD-GDH gene, can be performed by any known method depending on the intended form of mutation. That is, methods of bringing a chemical agent serving as a mutagen into contact with and allowing to act on the FAD-GDH gene or recombinant DNA having the gene integrated therein; ultraviolet irradiation methods; genetic engineering methods; or protein engineering methods, can be extensively used.

As the chemical agent serving as a mutagen in the above mutation treatment, hydroxylamine, N-methyl-N′-nitro-N-nitrosoguanidine, nitrous acid, sulfurous acid, hydrazine, formic acid or 5-bromouracil and the like can be mentioned.

Conditions for the contact and action can be determined depending on the type of chemical agent being used and the like and are not particularly limited as long as a desired mutation can actually be induced in the FAD-GDH gene. A desired mutation can be induced usually by allowing the chemical agent to be in contact or act on the gene, preferably at a chemical agent concentration of 0.5 to 12 M and at a reaction temperature of 20 to 80° C. for 10 minutes or more, and preferably 10 to 180 minutes. In the case of ultraviolet irradiation, the irradiation can be performed in accordance with the routine method mentioned above (Chemistry Today, p 24 to 30, 1989, June issue).

As a method employing a protein engineering procedure, in general, a method known as Site-Specific Mutagenesis can be used. Examples thereof include the Kramer method (Nucleic Acids Res., 12, 9441 (1984): Methods Enzymol., 154, 350 (1987): Gene, 37, 73 (1985)), the Eckstein method (Nucleic Acids Res., 13, 8749 (1985): Nucleic Acids Res., 13, 8765 (1985): Nucleic Acids Res, 14, 9679 (1986)) and the Kunkel method (Proc. Natl. Acid. Sci. U.S.A., 82, 488 (1985): Methods Enzymol., 154, 367 (1987)) and the like. Examples of specific methods for converting a nucleotide sequence in a DNA include methods using commercially available kits (e.g., Transformer Mutagenesis Kit, Clontech; EXOIII/Mung Bean Deletion Kit, manufactured by Stratagene; Quick Change Site Directed Mutagenesis Kit, manufactured by Stratagene).

Alternatively, a method known as a general Polymerase Chain Reaction can be used (Technique, 1, 11 (1989)). Incidentally, other than the above methods for modifying a gene, a FAD-GDH gene of interest can be directly synthesized using organic synthesis methods or enzyme synthesis methods.

The nucleotide sequence of DNA of the FAD-GDH gene selected by any method such as those mentioned above can be determined or confirmed by using, for example, multi-capillary DNA analysis system, CEQ2000 (manufactured by Beckman Coulter, Inc.).

### (Vector Having an FAD-GDH Gene Inserted Therein and Host Cell)

The FAD-GDH gene obtained as described above can be integrated into a vector such as a bacteriophage, a cosmid or a plasmid for use in transformation of prokaryote cells or eukaryote cells in accordance with a routine method, and the host cells corresponding to the vectors can be transformed or transduced by routine methods.

Examples of the prokaryotic host cells include microorganisms belonging to the genus Escherichia, such as E. coli K-12 strain, Escherichia coli BL21 (DE3), Escherichia coli JM109, Escherichia coli DH5α, Escherichia coli W3110 and Escherichia coli C600 (all manufactured by Takara Bio Inc.). These microbial cells are transformed or transduced to obtain host cells (transformants) having DNA introduced thereinto. As a method for transferring a recombinant vector to such a host cell, in the case where the host cell is a microorganism belonging to Escherichia coli, a method of transferring (introducing) recombinant DNA in the presence of calcium ions can be employed. Furthermore, an electroporation method may be used. Moreover, commercially available competent cells (for example, ECOS Competent Escherichia coli BL21 (DE3); manufactured by Nippon Gene Co., Ltd.) may be used.

An example of the eukaryotic host cell includes yeast. Examples of microorganisms classified as yeast include yeasts belonging to the genus Zygosaccharomyces, the genus Saccharomyces, the genus Pichia and the genus Candida. The gene to be inserted may contain a marker gene which enables selection of transformed cells. Examples of the marker gene include genes which compensate auxotrophy of a host cell, such as URA3 and TRP1. The gene to be inserted may desirably contain a promoter enabling expression of the gene of interest in a host cell or other regulatory sequences (for example, enhancer sequence, terminator sequence, polyadenylation sequence). Specific examples of the promoter include GAL1 promoter and ADH1 promoter. As methods for transforming yeast, known methods such as the method of using lithium acetate (MethodsMol. Cell. Biol., 5, 255-269 (1995)) as well as electroporation (J Microbiol Methods 55 (2003) 481-484) can be suitably used. However, the transformation method is not limited to these and any method including the spheroplast method and glass bead method can be used for transformation.

Other examples of the eukaryotic host cell include fungi such as those of the genus Aspergillus and the genus Tricoderma. The method for preparing a transformant of a fungus is not particularly limited and includes, for example, a method of inserting a gene encoding an FAD-GDH to a host fungus with routine methods such that the gene is expressed. More specifically, a DNA construct is prepared by inserting an FAD-GDH gene between an expression inducing promoter and a terminator; and then, a host filamentous fungus is transformed with the DNA construct containing the FAD-GDH gene to obtain transformants overexpressing the FAD-GDH gene. In the present specification, a DNA fragment consisting of an expression inducing promoter-FAD-GDH encoding gene-terminator, and a recombinant vector comprising said DNA fragment, produced to transform a host filamentous fungus, are collectively referred to as DNA constructs.

The method for inserting a gene encoding a FAD-GDH into a host filamentous fungus such that the gene is expressed is not particularly limited and includes, for example, a method of directly inserting the gene into the chromosome of a host organism by using homologous recombination; and a method of ligating the gene to a plasmid vector and introducing the vector to a host filamentous fungus.

In the method using homologous recombination, a DNA construct is inserted into the genome of the host filamentous fungus by ligating the DNA construct between sequences homologous to the upstream region and downstream region of a recombination site on the chromosome. By overexpressing the gene under control of its own high expression promotor in the host filamentous fungus, a transformant by self-cloning can be obtained. Examples of the high expression promoter include, but are not particularly limited to, the promoter region of TEF1 gene (tef1) serving as a translation elongation factor, the promoter region of α-amylase gene (amy) and the promoter region of an alkaline protease gene (alp).

In the method using a vector, a DNA construct is integrated into a plasmid vector used for transformation of filamentous fungi by routine methods and then the corresponding host filamentous fungus can be transformed (with the plasmid vector) using routine methods.

Such suitable vector-host system is not particularly limited as long as the FAD-GDH can be produced in the host filamentous fungus and includes, for example, pUC19 and filamentous fungus system, pSTA14 (Mol. Gen. Genet. 218, 99-104, 1989) and filamentous fungus system.

It is preferable to use the DNA construct by introducing the same into the chromosome of a host filamentous fungus. However, as an alternative method, the DNA construct can be integrated into an autonomous replicating vector (Ozeki et al. Biosci. Biotechnol. Biochem. 59, 1133 (1995)). In this manner, the DNA construct can be used without being introduced into the chromosome.

The DNA construct may comprise a marker gene which enables a transformed cell to be selected. Examples of the marker gene include, but are not particularly limited to, genes compensating auxotrophy of a host such as pyrG, niaD, adeA; and drug resistance genes against chemical agents such as pyrithiamine, hygromycin B and oligomycin. Further, the DNA construct preferably comprises a promoter enabling overexpression of the gene encoding the FAD-GDH in the host cell, a terminator and other regulatory sequences (for example, enhancer, polyadenylation sequence). Examples of the promoter include, but are not particularly limited to, suitable expression induction promoters and constitutive promoters, such as the tef1 promoter, alp promoter, amy promoter and the like. Examples of the terminator include, but are not particularly limited to, the alp terminator, amy terminator and tef1 terminator and the like.

In the DNA construct, if the DNA fragment containing the gene encoding the FAD-GDH to be inserted has a sequence having expression regulating function, then an expression regulatory sequence for the gene encoding the FAD-GDH need not be required. When transformation is carried out by a co-transformation method, the DNA construct need not have a marker gene in some cases.

One embodiment of a DNA construct is, for example, a DNA construct prepared by ligating the tef1 gene promoter, a gene encoding FAD-GDH, the alp gene terminator and the pyrG marker gene to the In-Fusion Cloning Site within the multiple cloning site of pUC19.

As a method for transforming filamentous fungi, methods known to those skilled in the art can appropriately be selected, for example, a protoplast PEG method can be used, in which a protoplast of a host filamentous fungus is prepared, and then, polyethylene glycol and calcium chloride are used (see, for example, Mol. Gen. Genet. 218, 99-104, 1989, JP Patent Publication (Kokai) No. 2007-222055A). As the culture medium for regenerating a transformed filamentous fungus, an appropriate culture medium is used depending on the host filamentous fungus to be used and the transformation marker gene. For example, if Aspergillus soya is used as the host filamentous fungus and pyrG gene is used as the transformation marker gene, the transformed filamentous fungus can be regenerated in Czapek-Dox minimal medium (Difco) containing for example, 0.5% agar and 1.2 M sorbitol.

To obtain, for example, the transformed filamentous fungus, the promoter of the gene encoding the FAD-GDH that the host filamentous fungus originally has in the chromosome may be substituted with a high expression promoter such as tef1, by using homologous recombination. In this case, a transformation marker gene such as pyrG is preferably inserted in addition to the high expression promoter. For example, for this purpose, a transformation cassette consisting of an upstream region of a gene encoding FAD-GDH-transformation marker gene-high expression promoter-whole or part of gene encoding FAD-GDH, can be used (see, Example 1 and FIG. 1 in JP Patent Publication (Kokai) No. 2011-239681A). In this case, the upstream region of a gene encoding FAD-GDH and the whole or part of the gene encoding FAD-GDH are used for homologous recombination. As the whole or part of the gene encoding FAD-GDH, a region containing the initiation codon up to a midstream region can be used. The length of the region suitable for homologous recombination is preferably 0.5 kb or more.

Whether the transformed filamentous fungus was produced or not can be confirmed by culturing the transformed filamentous fungus under conditions where FAD-GDH enzyme activity can be confirmed and then confirming the FAD-GDH activity in a culture obtained after culturing.

Further, whether the transformed filamentous fungus was produced or not can be confirmed by extracting chromosomal DNA from a transformed filamentous fungus, subjecting the chromosomal DNA to PCR using the chromosomal DNA as the template and confirming production of a PCR product that can be amplified if transformation took place.

For example, PCR is carried out by using a forward primer to the nucleotide sequence of the applied promoter in combination with a reverse primer to the nucleotide sequence of the transformation marker gene, and then, whether or not a product having the predicted length is obtained, is confirmed.

### (Method for Producing the FAD-GDH Enzyme)

The FAD-GDH enzyme may be produced by culturing a host cell capable of producing the FAD-GDH and obtained as mentioned above, expressing the FAD-GDH gene contained in the host cell, and then, isolating the FAD-GDH from the culture.

As the culture medium for culturing the host cell above, a culture medium prepared by adding one or more nitrogen sources such as yeast extract, tryptone, peptone, meat extract, corn steep liquor or soy or wheat bran steep liquor and one or more inorganic salts such as sodium chloride, primary potassium phosphate, secondary phosphate, magnesium sulfate, magnesium chloride, ferric chloride, ferric sulfate or manganese sulfate, and if necessary, further adding a sugar source and vitamins and the like, is used.

The initial pH of a culture medium is not limited; however the pH can be adjusted to 6 to 9 for example.

Culturing may be performed at a culture temperature of 10 to 42° C., preferably about 25° C., for 4 hours to one week, and further preferably performed at a culture temperature of about 25° C. for 4 hours to 5 days, in accordance with, e.g., aeration stirring deep culture, shaking culture or static culture.

After completion of culture, the FAD-GDH enzyme is recovered from the culture. This can be carried out by a known enzyme sampling means routinely used. For example, the fungus body can be subjected to a routine treatment such as a ultrasonication disruption treatment or a grinding treatment; or the FAD-GDH of the invention can be extracted by using a lytic enzyme such as lysozyme or Yatalase™. Alternatively, the fungus body can be shaken or allowed to stand in the presence of toluene or the like to cause cell lysis, and the FAD-GDH of the invention can be discharged out of the fungus body. Subsequently, the lysis solution is filtered or centrifuged and solid matter is removed and if necessary, nucleic acids are removed by streptomycin sulfate, protamine sulfate or manganese sulfate and the like. Then, to the resultant solution, ammonium sulfate, an alcohol or acetone and the like is added, the mixture is fractionated and the precipitate is collected to obtain a crude enzyme of the FAD-GDH.

The crude enzyme of the FAD-GDH can be further purified by any known means. A purified enzyme preparation can be obtained by a method appropriately selected from, for example, a gel filtration method using Sephadex, ultrogel or bio gel; an adsorption elution method using an ion exchanger; an electrophoretic method using, e.g., polyacrylamide gel; an adsorption elution method using hydroxyapatite; a sedimentation method such as a sucrose density gradient centrifugation method; an affinity chromatography method; and a fractionation method using, e.g., a molecular sieve membrane or a hollow fiber membrane, or by using these methods in combination. In this manner, a purified FAD-GDH enzyme preparation can be obtained.

In one embodiment, glucose dehydrogenase may be immobilized onto a solid phase. In one embodiment, the present disclosure provides a glucose measurement kit or a glucose measurement system (apparatus) that comprises the above described substrate specificity modifier and a solid phase onto which glucose dehydrogenase is immobilized. Examples of the solid phase include, but are not limited to, a bead or a particle, a polymer, and an electrode surface. For example, in one embodiment, glucose dehydrogenase may be immobilized onto an electrode. In one embodiment, the present disclosure provides a glucose measurement system (apparatus) that comprises the above described substrate specificity modifier and an electrode onto which glucose dehydrogenase is immobilized.

### (Method for Immobilizing Glucose Dehydrogenase)

The glucose dehydrogenase may be immobilized onto a solid phase by any known method. The glucose dehydrogenase may be immobilized onto a bead or a membrane, a carbon particle, a gold particle, a platinum particle, a polymer, or an electrode surface. Examples of the immobilization method include a method using a cross-linking reagent, an entrapment method in a polymer matrix, a coating method with a dialysis membrane, and methods of using a photo-crosslinkable polymer, a conductive polymer, a redox polymer, or the like. Glucose dehydrogenase may be immobilized in the polymer or may be immobilized by adsorption onto an electrode, or these methods may be used in combination. Typically, glucose dehydrogenase is immobilized onto a carbon electrode by using glutaraldehyde and then treated with a reagent having an amine group to block glutaraldehyde. In one embodiment, the glucose dehydrogenase substrate specificity modifier of the present disclosure may be present when glucose dehydrogenase is immobilized onto a solid phase.

In one embodiment, when glucose dehydrogenase immobilized onto an electrode is used or when glucose dehydrogenase is used without being immobilized, known mediators can be used as a mediator. Examples of the known mediators include phenazine compounds such as 1-methoxy-5-methylphenazinium methyl sulfate (mPMS), 5-methylphenazinium methyl sulfate (PMS), and 5-ethylphenazinium methyl sulfate (PES); phenothiazine compounds; ferricyanides such as potassium ferricyanide; ferrocene compounds such as ferrocene, dimethylferrocene, and ferrocene carboxylic acid; quinone compounds such as naphthoquinone, anthraquinone, hydroquinone, and pyrroloquinoline quinone; cytochrome compounds; viologen compounds such as benzyl viologen and methyl viologen; indophenol compounds such as dichlorophenolindophenol; ruthenium complexes such as hexaammineruthenium chloride; osmium complexes such as osmium-2,2′-bipyridine; and phenylenediamine compounds such as p-phenylenediamine, N-isopropyl-N′-phenyl-p-phenylenediamine (IPPD), N′,N-diphenyl-p-phenylenediamine (DPPD), N-(1,3-dimethylbutyl)-N′-phenyl-p-phenylenediamine, and N,N,N′,N′-tetramethyl-1,4-phenylenediamine. These mediators can also be used in a form modified with a polymer such as polyvinyl imidazole, polyethyleneimine, and polyacrylic acid. Furthermore, these mediators can be immobilized onto GDH by using, for example, a cross-linking reagent. Furthermore, these mediators can be immobilized onto an electrode by hydrophobic interaction with reference to JP Patent No. 6484741 and JP Patent No. 6484742. The entire contents of these documents are incorporated herein by reference. Furthermore, these mediators can be immobilized onto an electrode after the electrode surface was activated by, for example, an acid treatment. For example, a material such as carbon, gold, and platinum can be used as an electrode.

The final concentration of the mediator to be added to a sample solution is not particularly limited and may range, for example, from 1 pM to 1M, from 1 pM to 100 mM, from 1 pM to 20 mM, from 1 pM to 10 mM, from 1 pM to 5 mM, from 2 pM to 1 mM, from 3 pM to 800 μM, from 4 pM to 600 μM, from 5 pM to 500 μM, from 6 pM to 400 μM, from 7 pM to 300 μM, from 8 pM to 200 μM, from 9 pM to 100 μM, and from 10 pM to 50 μM. In relation to this, the order in which the mediator and other reagents are added is not restricted, and they may be added simultaneously or sequentially.

In one embodiment, the length of a redox reaction or the length of electrochemical measurement can be set to 60 minutes or less, 30 minutes or less, 10 minutes or less, 5 minutes or less, or 1 minute or less. Alternatively, when a device involving measurement over a long period of time, for example an enzyme sensor or an electrical cell is used, the length of the redox reaction can be set to 60 minutes or more, 120 minutes or more, 1 day or more, 2 days or more, 3 days or more, 1 week or more, 2 weeks or more, or 3 weeks or more.

Unless otherwise specified, the glucose dehydrogenase comprised in the composition or the glucose dehydrogenase immobilized onto the electrode is a purified glucose dehydrogenase enzyme.

(Measurement of the Activity of Glucose Dehydrogenase)

Measurement of the activity of glucose dehydrogenase is explained. GDH (EC 1.1.99.10) catalyzes the reaction of oxidizing a hydroxyl group of glucose to produce glucono-δ-lactone. In this regard, an electron acceptor accepts an electron and becomes a reduced-form electron acceptor. The activity of the GDH can be measured based on this principle of action and by using, for example, the following measurement system which employs phenazine methosulfate (PMS) and 2,6-dichloroindophenol (DCIP) as electron acceptors.

D-glucose+PMS (oxidized form)→D-glucono-δ-lactone+PMS (reduced form) (Reaction 1)

PMS (reduced form)+DCIP (oxidized form)→PMS (oxidized form)+DCIP (reduced form) (Reaction 2)

More specifically, first, in (Reaction 1), as oxidation of D-glucose proceeds, PMS (reduced form) is generated. Subsequently, (Reaction 2) proceeds, in which as oxidation of PMS (reduced form) proceeds, DCIP is reduced. The degree of disappearance of “DCIP (oxidized form)” is detected as an amount of change in absorbance at a wavelength of 600 nm and the enzyme activity can be determined based on this amount of change in absorbance.

The activity of the FAD-GDH can be measured with the following procedure. First, 2.05 mL of 100 mM phosphate buffer (pH 7.0), 0.6 mL of 1M D-glucose solution and 0.15 mL of 2 mM DCIP solution are mixed and the mixture is kept at a temperature of 37° C. for 5 minutes. Then, to the mixture, 0.1 mL of a 15 mM PMS solution and 0.1 mL of the enzyme sample solution are added to initiate the reaction. Absorbance is measured at the initiation of the reaction and over time. The decrease (AA600) of absorbance at 600 nm per minute as the enzymatic reaction proceeds is obtained and GDH activity is calculated with the following formula. Herein, 1 U of GDH activity is defined as the amount of the enzyme required for reducing 1 μmol of DCIP at 37° C. in the presence of D-glucose (concentration 200 mM) per minute.

$$GDH Activity ( U \text{/} mL ) = \frac{- ( Δ A 600 - Δ A 60 {0}_{b l a n k} ) \times 3.0 \times d F}{16.3 \times 0.1 \times 1.0} \qquad [ Formula 1 ]$$

Note that, in the formula, the numerical value 3.0 represents the amount (mL) of liquid (reaction reagent+enzyme reagent); the numerical value 16.3 represents the millimolar molecular extinction coefficient (cm²/μmol) under this activity measurement condition; the numerical value 0.1 represents the amount of the enzyme solution (mL); the numerical value 1.0 represents the optical path length of the cell (cm), AA600blank represents the decrease in absorbance at 600 nm per minute in the case where the reaction is initiated by adding a 100 mM phosphate buffer (pH 7.0) in place of the enzyme sample solution; and the reference symbol, df represents the dilution factor.

The glucose measurement kit of the present disclosure comprises the substrate specificity modifier in an amount sufficient for at least one assay. Typically, the glucose measurement kit of the present disclosure comprises, in addition to the substrate specificity modifier, glucose dehydrogenase, a buffer necessary for the assay, a glucose substrate standard solution for generating a calibration curve, and instructions. The substrate standard solution may be a glucose standard solution. Furthermore, to evaluate the reactivity of glucose dehydrogenase to maltose, a maltose substrate standard solution of a known concentration can be used.

In one embodiment, the glucose measurement kit of the present disclosure comprises the substrate specificity modifier and the glucose dehydrogenase as a single reagent. In another embodiment, the glucose measurement kit of the present disclosure comprises the substrate specificity modifier and the glucose dehydrogenase as separate reagents. In another embodiment, the glucose dehydrogenase may be immobilized onto an electrode, and the glucose measurement kit of the present disclosure used for such an electrode comprises the substrate specificity modifier as a single reagent. Note that the term “single reagent” herein does not mean that the reagent does not contain substances other than the substrate specificity modifier. The substrate specificity modifier may be in a solution state or a dry state, for example, powders.

Examples of colorimetric assays include measurement of glucose concentration. Measurement of glucose concentration in the case of a colorimetric measurement can, for example, be carried out as follows. In the reaction layer, a liquid or solid state composition containing glucose dehydrogenase (GDH) and at least one or more substance selected from the group consisting of N-(2-acetamide)imide diacetate (ADA), bis(2-hydroxyethyl)iminotris(hydroxymethyl) methane (Bis-Tris), sodium carbonate and imidazole, as a reaction accelerator, are retained. A pH buffer and a coloring reagent (color changing reagent) can be added according to need. To this, a sample containing glucose is added and allowed to react for a predetermined time. During this time period, the absorbance of a reduced dye or dye generated by polymerization upon receiving an electron directly from the GDH, at a wavelength corresponding to the maximum absorption wavelength, is monitored. The glucose concentration in a sample can be computed from the rate of change of absorbance per time if a rate method is employed; or computed from a change of absorbance up to the time point where glucose in the sample is completely oxidized if an endpoint method is employed, based on a calibration curve prepared in advance using a standard-concentration glucose solution.

Examples of the coloring reagent (color changing reagent) to be used in this method include 2,6-dichloroindophenol (DCIP), which can be added as an electron acceptor and the amount of glucose can be determined by monitoring a decrease in absorbance at 600 nm. Glucose concentration can be computed by adding nitrotetrazolium blue (NTB) as a coloring reagent and measuring the absorbance at 570 nm to determine the amount of diformazan generated. In this regard and needless to mention, the coloring reagent (color changing reagent) to be used herein are not limited to the above.

### (Enzyme Sensor)

In one embodiment, glucose can be detected or measured by an enzyme sensor. The enzyme sensor may comprise an electrode onto which glucose dehydrogenase is immobilized. Furthermore, the electrode may have the above described mediator adsorbed thereonto. Examples of the electrode for the enzyme sensor include a carbon electrode, a gold electrode, and a platinum electrode and the glucose dehydrogenase may be applied or immobilized onto such an electrode. Furthermore, metal microparticles may be included as an electroconductive material, the metal microparticles containing at least one element selected from the group consisting of Co, Pd, Rh, Ir, Ru, Os, Re, Ni, Cr, Fe, Mo, Ti, Al, Cu, V, Nb, Zr, Sn, In, Ga, Mg, Pb, Au, Pt, and Ag and such metal microparticles may be alloy microparticles or plated microparticles. Examples of the carbon include a carbon nanotube, carbon black, graphite, a fullerene, and derivatives thereof. In one embodiment, the enzyme sensor may be a glucose sensor. Thus, in one embodiment, the present disclosure provides a glucose detection method or a glucose measurement method that uses the substrate specificity modifier and the glucose sensor as described above. The glucose sensor can be used for consecutive glucose measurement and/or continuous glucose monitoring.

The composition or reagent comprising the substrate specificity modifier of the present disclosure can be applied, along with an electrode onto which a glucose dehydrogenase is immobilized, or with an enzyme sensor, to various electrochemical measurements by using, a potentiostat, a galvanostat and the like. Examples of the electrochemical measurement method include, various methods such as amperometry, for example chronoamperometry, potential step chronoamperometry, voltammetry, such as cyclic voltammetry, differential pulse voltammetry, potentiometry and coulometry and the like. For example, in glucose measurement, by using the amperometric method and measuring the current when glucose is reduced, it is possible to calculate the glucose concentration of a sample. The voltage to be applied varies depending on the conditions and setting of the apparatus and can be set to be, for example, −1000 mV to +1000 mV (vs. Ag/AgCl).

A printed electrode can also be used as an electrode. This can reduce the amount of solution required for measurement. The electrode may be formed on an insulating substrate. Specifically, the electrode may be formed on the substrate by a printing technique such as a photolithographic technique, screen printing, gravure printing, and flexographic printing. Examples of the materials for the insulating substrate include, silicone, glass, ceramics, polyvinyl chloride, polyethylene, polypropylene, and polyester; and those highly resistant to various solvents and/or agents can be used. Carbon cloth, carbon paper, or Buckypaper can also be used as a base material for the electrode. The area of a working electrode can be set depending on a desired response current. For example, in one embodiment, the area of the working electrode can be set to 1 mm² or more, 1.5 mm² or more, 2 mm² or more, 2.5 mm² or more, 3 mm² or more, 4 mm² or more, 5 mm² or more, 6 mm² or more, 7 mm² or more, 8 mm² or more, 9 mm² or more, 10 mm² or more, 12 mm² or more, 15 mm² or more, 20 mm² or more, 30 mm² or more, 40 mm² or more, 50 mm² or more, 1 cm² or more, 2 cm² or more, 3 cm² or more, 4 cm² or more, or 5 cm² or more, for example 10 cm² or more. In one embodiment, the area of the working electrode can be set to 10 cm² or less or 5 cm² or less, for example 1 cm² or less. The above may also apply to a counter electrode. Furthermore, a carbon nanotube, graphene, Ketjenblack, and the like can be immobilized onto the working electrode to increase an apparent surface area. This may increase the apparent surface area by 10 times or more, 50 times or more, 100 times or more, or 1000 times or more.

In one embodiment, the present disclosure provides a screening method for a glucose dehydrogenase substrate specificity modifier. This method comprises the steps of:

i) providing a glucose dehydrogenase;
ii) determining the ratio (Mal/Glu) of the reactivity of the glucose dehydrogenase to maltose (Mal) relative to the reactivity thereof to glucose (Glu);
iii) contacting the glucose dehydrogenase described above in i) with a candidate substance, and then determining the ratio (Mal/Glu) of the reactivity of the glucose dehydrogenase to maltose (Mal) relative to the reactivity thereof to glucose (Glu) in the presence of the candidate substance;
iv) comparing the ratio (Mal/Glu) obtained in ii) with the ratio (Mal/Glu) obtained in iii) in the presence of the candidate substance; and
v) identifying the candidate substance as a glucose dehydrogenase substrate specificity modifier if the ratio (Mal/Glu) obtained in iii) in the presence of the candidate substance is altered compared to the ratio (Mal/Glu) obtained in ii). The candidate substance may be a low molecular weight compound that is a glucose analog.

In one embodiment, the present disclosure provides a method for confirming the effective concentration of a glucose dehydrogenase substrate specificity modifier. This method comprises the steps of:

i) providing a glucose dehydrogenase and glucose dehydrogenase substrate specificity modifiers at concentration 1, concentration 2, and up to concentration n (n is a natural number);
ii) individually determining the ratio (Mal/Glu) of the reactivity of the glucose dehydrogenase to maltose (Mal) relative to the reactivity thereof to glucose (Glu) in the presence of the glucose dehydrogenase substrate specificity modifiers at concentration 1, concentration 2, and up to concentration n; and
iii) comparing the ratios (Mal/Glu) for respective concentrations to confirm the effective concentration of the glucose dehydrogenase substrate specificity modifier. For example, in one embodiment, when the ratio Mal/Glu for a specific concentration n is substantially the same as the ratio Mal/Glu in the absence of the glucose dehydrogenase substrate specificity modifier, then the lower limit of the effective concentration of the glucose dehydrogenase substrate specificity modifier may be set to be the specific concentration n or a concentration higher than the same. In one embodiment, when the ratio Mal/Glu for a specific concentration n is substantially the same as the ratio Mal/Glu obtained when the glucose dehydrogenase substrate specificity modifier was added at concentration n−1, then the upper limit of the effective concentration of the glucose dehydrogenase substrate specificity modifier may be set to be the specific concentration n or a concentration lower than the same. Note that concentration n is a concentration higher than concentration n−1.

In one embodiment, the present disclosure provides a method for producing a reagent for glucose measurement or a composition for glucose measurement, comprising the step of formulating (incorporating) a glucose dehydrogenase substrate specificity modifier identified by the above described screening method into the reagent for glucose measurement or the composition for glucose measurement. In another embodiment, the present disclosure also provides a reagent for glucose measurement, comprising glucose dehydrogenase and a glucose dehydrogenase substrate specificity modifier identified by the above described screening method.

The ratio (Mal/Glu) of the reactivity of the glucose dehydrogenase to maltose (Mal) relative to the reactivity thereof to glucose (Glu) can be determined by the following procedures. Onto a printed carbon electrode, N-isopropyl-N′-phenyl-p-phenylenediamine (IPPD) is immobilized by adsorption and a glucose dehydrogenase is immobilized by cross-linking using glutaraldehyde, wherein the glucose dehydrogenase is, for example, a glucose dehydrogenase from a microorganism of the genus Mucor or a glucose dehydrogenase from a microorganism of the genus Aspergillus. Chronoamperometry is performed by using a three-electrode system composed of a working electrode, a counter electrode, and a reference electrode and applying a potential of +250 mV (Ag/AgCl). Once the current value has become sufficiently constant, maltose is added to a final concentration of 1 mM in phosphate-buffered saline (PBS) (for example, maltose is added 100 seconds after the start of measurement). Next, once the current value has become sufficiently constant again after the addition of maltose, glucose is added to a final concentration of 1 mM in phosphate-buffered saline (PBS) (for example, glucose is added 100 seconds after the addition of maltose). The substrate specificity of glucose dehydrogenase can be evaluated by measuring the reactivity thereof to maltose when the reactivity thereof to glucose is considered as 100%. In the present specification, the ratio of the reactivity to maltose (Mal) relative to the reactivity to glucose (Glu) may be denoted as Mal/Glu. When the reactivity is evaluated based on the response current(s), the ratio of the reactivity may be expressed as the ratio of the response current(s).

For example, when the Mal/Glu of a glucose dehydrogenase is 0.50% in the absence of a glucose dehydrogenase substrate specificity modifier and the Mal/Glu of the glucose dehydrogenase as determined under the same measurement conditions except for the presence of a glucose dehydrogenase substrate specificity modifier is less than 0.50%, then it may be concluded that the reactivity of the glucose dehydrogenase to maltose was reduced and the substrate specificity thereof was modified. Such a glucose dehydrogenase substrate specificity modifier may reduce the effect of maltose on glucose measurement. In this context, the relative value Mal/Glu of a glucose dehydrogenase in the absence of a glucose dehydrogenase substrate specificity modifier may be defined as 100%. In this case, when the Mal/Glu of the glucose dehydrogenase as determined under the same measurement conditions except for the presence of a glucose dehydrogenase substrate specificity modifier is less than 100%, it may be concluded that the reactivity of the glucose dehydrogenase to maltose was reduced and the substrate specificity thereof was modified. On the other hand, when the Mal/Glu of the glucose dehydrogenase in the presence of a glucose dehydrogenase substrate specificity modifier is 100% or more, it may be concluded that the reactivity of the glucose dehydrogenase to maltose was increased and the substrate specificity thereof was modified.

In one embodiment, when the Mal/Glu of a glucose dehydrogenase in the absence of a glucose dehydrogenase substrate specificity modifier is considered as 100%, the Mal/Glu value (ratio) of the glucose dehydrogenase in the presence of a glucose dehydrogenase substrate specificity modifier of the present disclosure may be 99% or less, 98% or less, 97% or less, 96% or less, 95% or less, 94% or less, 93% or less, 92% or less, 91% or less, 90% or less, 89% or less, 88% or less, 87% or less, 86% or less, 85% or less, 84% or less, 83% or less, 82% or less, 81% or less, 80% or less, 79% or less, 78% or less, 77% or less, 76% or less, 75% or less, 74% or less, 73% or less, 72% or less, 71% or less, 70% or less, 69% or less, 68% or less, 67% or less, 66% or less, 65% or less, 64% or less, 63% or less, 62% or less, 61% or less, 60% or less, 59% or less, 58% or less, 57% or less, 56% or less, 55% or less, 54% or less, 53% or less, 52% or less, 51% or less, 50% or less, 49% or less, 48% or less, 47% or less, 46% or less, 45% or less, 44% or less, 43% or less, 42% or less, 41% or less, 40% or less, 39% or less, 38% or less, 37% or less, 36% or less, 35% or less, 34% or less, 33% or less, 32% or less, 31% or less, 30% or less, 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less, for example 1% or less, for example substantially 0%, for example 0%; for example, 99% to 0%, 99% to 1%, 98% to 2%, 97% to 3%, 96% to 4%, 95% to 5%, 94% to 6%, 93% to 7%, 92% to 8%, 91% to 9%, 90% to 10%, 89% to 11%, 88% to 12%, 87% to 13%, 86% to 14%, 85% to 15%, 84% to 16%, 83% to 17%, 82% to 18%, 81% to 19%, 80% to 20%, 79% to 21%, 78% to 22%, 77% to 23%, 76% to 24%, 75% to 25%, 74% to 26%, 73% to 27%, 72% to 28%, 71% to 29%, 70% to 30%, 69% to 31%, 68% to 32%, 67% to 33%, 66% to 34%, 65% to 35%, 64% to 36%, 63% to 37%, 62% to 38%, 61% to 39%, 60% to 40%, 59% to 41%, 58% to 42%, 57% to 43%, 56% to 44%, 55% to 45%, 54% to 46%, 53% to 47%, 52% to 48%, or 51% to 49%, for example 50%.

In one embodiment, when the Mal/Glu of a glucose dehydrogenase in the absence of a glucose dehydrogenase substrate specificity modifier is considered as 100%, the Mal/Glu value of the glucose dehydrogenase in the presence of a glucose dehydrogenase substrate specificity modifier of the present disclosure may be 110% or more, 120% or more, 130% or more, 140% or more, 150% or more, 160% or more, 170% or more, 180% or more, 190% or more, 200% or more, 220% or more, 240% or more, 260% or more, 280% or more, 300% or more; for example, 110% to 300%, 120% to 280%, 130% to 260%, 140% to 240%, 150% to 220%, 160% to 200%, for example 170% to 190%. Unless otherwise specified, the numerical range in the present disclosure shall include both the upper limit and the lower limit (for example, the numerical range from a to b means a range not less than a and not more than b). Furthermore, the present disclosure shall encompass all combinations of the exemplary upper and lower limits of the numerical range.

Without wishing to be bound to any specific theory, the ability of the glucose dehydrogenase substrate specificity modifier of the present disclosure to modify the substrate specificity of GDH is believed to be based on the following mechanism of action. Specifically, glucose dehydrogenase is known to use its substrate pocket to recognize and act on glucose, and a compound having a chemical structure similar to that of glucose (glucose analog) is believed to enter the substrate pocket ahead of other substances, thereby preventing a contaminant sugar (coexisting sugar) other than glucose, for example maltose from entering the substrate pocket. Incidentally, while glucose is a C6 compound and although glycerol is a C3 compound, glycerol may be considered structurally identical to a part of glucose and since glycerol also exhibited an action of modifying the substrate specificity of glucose dehydrogenase, it is believed that glycerol enters the substrate pocket or interacts with the substrate pocket.

According to the present disclosure, the substrate specificity of glucose dehydrogenase can be modified. The present disclosure may be combined with different methods of modifying the substrate specificity of a glucose dehydrogenase. For example, a glucose dehydrogenase having a high substrate specificity can be searched for and a new glucose dehydrogenase so discovered can be used in combination with the GDH substrate specificity modifier of the present disclosure. As another example, the substrate specificity of a GDH may be modified and the modified GDH mutant can be used in combination with the GDH substrate specificity modifier of the present disclosure.

In one embodiment, the glucose dehydrogenase substrate specificity modifier used for the glucose measurement method of the present disclosure may comprise one or more compounds selected from the group consisting of L-gulose, D-iditol, L-iditol, D-glucal, sorbitol, ribitol, and trehalose, as well as D-mannitol, xylitol, and glycerol; and the glucose dehydrogenase may be an FAD-GDH from a microorganism of the genus Mucor, for example a GDH from Mucor prainii (MpGDH), and the FAD-GDH may or may not be immobilized onto an electrode.

In one embodiment, the glucose dehydrogenase substrate specificity modifier used for the glucose measurement method of the present disclosure may comprise one or more compounds selected from the group consisting of L-gulose, D-iditol, D-glucal, sorbitol, ribitol, and trehalose, as well as D-mannitol, xylitol, and glycerol; and the glucose dehydrogenase may be an FAD-GDH from a microorganism of the genus Mucor, for example Mucor RD056860 GDH (MrdGDH), and the FAD-GDH may or may not be immobilized onto an electrode.

In one embodiment, the glucose dehydrogenase substrate specificity modifier used for the glucose measurement method of the present disclosure may comprise one or more compounds selected from the group consisting of L-gulose, D-iditol, D-glucal, ribitol, and trehalose, as well as D-mannitol, xylitol, and glycerol; and the glucose dehydrogenase may be an FAD-GDH from a microorganism of the genus Aspergillus, for example an FAD-GDH from Aspergillus oryzae or Aspergillus terreus, or GLD1, and the FAD-GDH may or may not be immobilized onto an electrode.

The substrate specificity modifier of the present disclosure can be used to modify the substrate specificity of glucose dehydrogenase. This can reduce the effect of maltose when the glucose concentration of a sample is measured using glucose dehydrogenase, enabling more accurate measurement of the glucose concentration. This may be useful for blood glucose control for diabetic patients.

### (Electrode of the Present Disclosure)

In one embodiment, the present disclosure provides an electrode comprising a glucose dehydrogenase substrate specificity modifier. In one embodiment, the electrode may be an anode electrode. In one embodiment, the anode electrode of the present disclosure comprises glucose dehydrogenase. In one embodiment, the glucose dehydrogenase may be immobilized on an electrode of an electrical cell. In one embodiment, the anode electrode of the present disclosure can be combined with a cathode electrode and a resistance to provide an electrical cell. In one embodiment, the present disclosure provides a method for generating electricity by using the above described electrical cell. In another embodiment, an electrical cell is provided, wherein the cell comprises the glucose dehydrogenase substrate specificity modifier of the present disclosure.

### (Fuel Cell of the Present Disclosure)

In one embodiment, the present disclosure provides an anode or a cathode for a fuel cell and a fuel cell comprising the anode or the cathode. This fuel cell comprises the glucose dehydrogenase substrate specificity modifier of the present disclosure. In one embodiment, the present disclosure provides a method for generating electricity by using an electrical cell comprising the above described glucose dehydrogenase substrate specificity modifier; and a method for generating electricity in the presence of a glucose dehydrogenase substrate specificity modifier, wherein a glucose dehydrogenase is immobilized onto an anode electrode and a substrate for the glucose dehydrogenase such as glucose is used as a fuel.

In one embodiment, the fuel cell of the present disclosure comprises the above described glucose dehydrogenase substrate specificity modifier, an anode or a cathode, a fuel tank, a cathode, an anode having a glucose dehydrogenase, and an electrolyte. An artificial electron mediator may be adsorbed on the anode or the cathode. Examples of the artificial electron mediator include, but are not limited to, the above described known mediators and the phenylenediamine compounds described in JP Patent No. 6484741 and JP Patent No. 6484742. Furthermore, the fuel cell of the present disclosure can have a load resistance placed between the anode and the cathode if necessary, and may be equipped with wiring therefor. In one embodiment, the load resistance is a component of the fuel cell of the present disclosure. In one embodiment, the load resistance is not a component of the fuel cell of the present disclosure, and the fuel cell of the present disclosure is configured such that the cell can be connected to an appropriate load resistance. In the fuel cell of the present disclosure, an oxidoreductase (GDH) constitutes a part of the anode. For example, the oxidoreductase may be in the vicinity of or in contact with, immobilized on, or adsorbed onto the anode. The fuel tank contains a compound used as a substrate for the oxidoreductase immobilized on the electrode. For example, when a glucose dehydrogenase is immobilized on the electrode, the fuel may be glucose. In one embodiment, the fuel cell of the present disclosure may have an ion exchange membrane to separate the anode and the cathode. The ion exchange membrane may have pores of 1 nm to 20 nm. The anode may be a common electrode such as a carbon electrode. For example, an electrode made of conductive carbonaceous substance such as carbon black, graphite, and activated carbon, or an electrode made of metal such as gold, platinum and the like can be used. Specific examples thereof include carbon paper, carbon cloth, glassy carbon, a carbon nanotube, and HOPG (highly oriented pyrolytic graphite) and the like. Examples of the paired cathode include an electrode in which an electrocatalyst commonly used in the fuel cell, such as platinum or platinum alloy, is supported on an electric conductor composed of carbonaceous material such as carbon black, graphite, and activated carbon, or gold, platinum, or the like; or an electric conductor composed of the electrocatalyst itself, such as platinum and platinum alloy. These can be used as the cathode electrode in a manner such that an oxidant (for example, a cathode-side substrate, oxygen and the like) can be supplied to the electrocatalyst.

In another embodiment, a substrate-reducing enzyme electrode may be used as a cathode to be paired with the anode as described above composed of a substrate-oxidizing enzyme electrode. Examples of the oxidoreductase that reduce oxidants include known enzymes such as a laccase and bilirubin oxidase. When an oxidoreductase is used as a catalyst to reduce the oxidant, a known electron-transferring mediator may be used as needed. Examples of the oxidant include oxygen and the like.

In one embodiment, an oxygen selective membrane (for example, a dimethylpolysiloxane membrane) can be placed around the cathode electrode to avoid the effects of impurities (for example, ascorbic acid, uric acid and the like) that interfere with the electrode reaction at the cathode.

The method of the present disclosure for generating electricity comprises the step of supplying a fuel to an anode having an oxidoreductase, the fuel being a compound serving as a substrate for the oxidoreductase. When the fuel is supplied to the anode comprising the oxidoreductase, the substrate is oxidized and an electron is generated at the same time. The oxidoreductase transfers the generated electron to an electron-transferring mediator such as a phenylenediamine compound, which mediates the electron transfer between the oxidase and the electrode. Then, the electron-transferring mediator transfers the electron to a conductive substrate (the anode electrode). The electron travels from the anode electrode to the cathode electrode through wiring (external circuitry), thereby generating an electrical current.

Proton (H⁺) generated in the process described above travels through the electrolyte solution to the cathode electrode. Then, at the cathode electrode, the proton that has traveled from the anode through the electrolyte solution, the electron that has traveled from the anode side through the external circuit, and the oxidant such as oxygen and hydrogen peroxide (a cathode-side substrate) react to produce water. This reaction can be utilized to generate electricity.

In one embodiment, glucose in a living body can be used as a fuel for the anode. In this case, if the glucose dehydrogenase reacts with maltose in the living body, an electrical current larger than expected may flow. Thus, to control the electrical current, it is believed that a substrate specificity acting only on glucose is required for the glucose dehydrogenase. The substrate specificity modifier of the present disclosure can also be utilized for such purposes.

In several descriptions of the present disclosure, the following expression was used: the ratio (Mal/Glu) of the reactivity of a glucose dehydrogenase to maltose relative to the reactivity thereof to glucose can be “altered.” In this context, “alteration” may encompass “increase” and “reduction.” For example, to reduce the effect of maltose on measuring a glucose concentration, it is preferable to reduce the ratio (Mal/Glu). Furthermore, for example when glucose dehydrogenase is used for a fuel cell fueled by a mixture of glucose and maltose, to obtain a higher current value, it is preferable to increase the ratio (Mal/Glu). Accordingly, in one embodiment, the present disclosure provides not only a method for reducing the ratio (Mal/Glu) of glucose dehydrogenase but also a method for increasing the ratio (Mal/Glu), as well as a substrate specificity modifier therefor. In these embodiments, “alterations” in respective descriptions shall be read as “reduction” or “increase.” According to the present disclosure, the above described substrate specificity modifier can be used to alter the substrate specificity of a GDH depending on the use without genetically modifying the glucose dehydrogenase.

In one embodiment, the present disclosure provides a method of modifying the substrate specificity of glucose dehydrogenase by using a glucose dehydrogenase substrate specificity modifier, which is a glucose analog and a low molecular weight compound. In another embodiment, the present disclosure provides use of a glucose dehydrogenase substrate specificity modifier for modifying the substrate specificity of glucose dehydrogenase, wherein the modifier is a glucose analog and a low molecular weight compound. In another embodiment, the present disclosure provides a glucose dehydrogenase substrate specificity modifier for use in modifying the substrate specificity of glucose dehydrogenase, wherein the modifier is a glucose analog and a low molecular weight compound. In one embodiment, the method of the present disclosure does not involve any medical practice.

### EXAMPLES

The present invention will be further illustrated by way of the following Examples. However, the technical scope of the present invention is not limited by these Examples in any way.

### Example 1

Introduction of a GDH Gene from a Microorganism of the Genus Mucor to a Host and Confirmation of GDH Activity

A GDH from a microorganism of the genus Mucor (MpGDH) is described in JP Patent No. 4648993. The amino acid sequence thereof is shown in SEQ ID NO: 1 and the nucleotide sequence thereof is shown in SEQ ID NO: 2. The nucleotide sequence (SEQ ID No: 4) that codes for MrdGDH having the amino acid sequence of SEQ ID No: 3 was entirely synthesized by reference to the disclosure of JP Patent Publication (Kokai) No. 2013-176363A. The target gene, an MpGDH gene or an MrdGDH gene, was inserted into the multiple cloning site of plasmid pUC19 using routine methods to generate a DNA construct. More specifically, a pUC19 linearized vector provided with In-Fusion HD Cloning Kit (Clontech Laboratories, Inc.) was used as the pUC19 vector. Into the In-Fusion Cloning Site present in the multiple cloning site of the pUC19, the MpGDH gene or the MrdGDH gene was ligated using the above described In-Fusion HD Cloning Kit according to the protocol included with the kit and the plasmid construct (pUC19-MpGDH or pUC19-MrdGDH) was obtained.

These genes were expressed in an Aspergillus microorganism (Aspergillus sojae) and their GDH activities were evaluated.

Specifically, to obtain an MpGDH or an MrdGDH, Double-joint PCR (Fungal Genetics and Biology, 2004, Vol. 41, p. 973-981) was performed using a GDH gene to construct a cassette comprising a 5′ arm region-pyrG gene-TEF1 promoter gene-flavin binding GDH gene-3′ arm region. This cassette was used to transform a pyrG disrupted strain derived from Aspergillus sojae NBRC4239 strain (a strain lacking a 48 bp region upstream of the pyrG gene, 896 bp of the code region thereof, and a 240 bp region downstream thereof) by the following procedures. In this context, the pyrG gene is a uracil-auxotrophic marker. Conidia of the pyrG disrupted strain derived from the Aspergillus sojae NBRC4239 strain were inoculated into 100 ml of polypeptone dextrin liquid medium containing 20 mM uridine in a 500 ml Erlenmeyer flask. After shaking culture at 30° C. for about 20 hours, fungus bodies were collected. Protoplasts were prepared from the collected fungus bodies. The obtained protoplasts and 20 μg of DNA construct comprising the inserted target gene were used to carry out transformation by the protoplast PEG method. Then, incubation was carried out using a Czapek-Dox minimal medium (Difco Laboratories, Inc.; pH 6) containing 0.5% (w/v) agar and 1.2 M sorbitol at 30° C. for 5 days or more, and transformed Aspergillus sojae, which exhibited a colony forming ability, was obtained.

The obtained transformed Aspergillus sojae can grow on a uridine-free medium because the pyrG gene, which is a gene that complements uridine auxotrophy, has been introduced thereinto. This enabled selecting the transformed Aspergillus sojae as a strain having the target gene introduced thereinto. The obtained fungal strains were subjected to PCR to confirm and select a transformant of interest.

Transformants of Aspergillus sojae transformed with the MpGDH gene or the MrdGDH gene were used for to produce each of the GDHs.

Conidia of each fungal strain were inoculated into 40 ml of DPY liquid medium (1% (w/v) polypeptone, 2% (w/v) dextrin, 0.5% (w/v) yeast extract, 0.5% (w/v) KH₂PO₄, 0.05% (w/v) MgSO₄.7H₂O; pH unadjusted) in a 200 ml Erlenmeyer flask, and subjected to shaking culture at 160 rpm for 4 days at 30° C. Then, the fungus bodies were filtered out from the culture after cultivation. The obtained supernatant fraction of the culture medium was concentrated to 10 mL and desalted using Amicon Ultra-15, 30K NMWL (manufactured by Millipore Corporation), and the medium was replaced with 20 mM potassium phosphate buffer (pH 6.5) containing 150 mM NaCl. Subsequently, the mixture was applied onto HiLoad 26/60 Superdex 200 pg column (manufactured by GE Healthcare) equilibrated with 20 mM potassium phosphate buffer (pH 6.5) containing 150 mM NaCl and eluted with the same buffer. A fraction exhibiting GDH activity was collected to obtain a purified MpGDH preparation. A purified MrdGDH preparation was obtained in the same manner. These enzymes are in a state bound to FAD via the FAD binding site(s) thereof (holoenzyme).

### Example 2

The Effect of a Substrate Specificity Modifier when an Immobilized Enzyme is Used

10 μL of 12 mg/ml MpGDH purified preparation, 10 μL of 15 mM each glucose analog, and 10 μL of 20 mM potassium phosphate buffer were mixed and allowed to stand for 2 hours or longer. N-isopropyl-N′-phenyl-p-phenylenediamine (IPPD) was immobilized by adsorption onto screen-printed electrodes (manufactured by DropSens, Product number: DRP-C110) and 12 μg of each MpGDH was applied to the electrodes and allowed to dry at room temperature. Lastly, the electrodes were placed in 25% glutaraldehyde vapor and the resulting electrodes were washed with pure water to obtain IPPD.MpGDH-immobilized electrodes. Next, the electrode was connected to an ALS electrochemical analyzer 814D via a dedicated connector (DRP-CAC). Then, the IPPD.GDH-immobilized electrode, an Ag/AgCl reference electrode, and a platinum counter electrode were immersed in PBS and chronoamperometric measurement was carried out. The applied voltage was set to +250 mV (Ag/AgCl). Once the current value had become sufficiently constant (about 100 seconds after the start of measurement), maltose was added to a final concentration of 1 mM in PBS. Next, subsequently to the addition of the maltose solution, once the current value had become sufficiently constant again (about 100 seconds after the addition of maltose), a glucose solution was added to a final concentration of 1 mM. The difference in the response current before and after the addition of maltose was recorded as the response current value of maltose, and the difference in the response current before and after the addition of glucose was recorded as the response current value of glucose. The value calculated by dividing the response current value of maltose by the response current value of glucose was expressed as maltose/glucose (%). Results are shown in Table 1. Sorbitol, ribitol, L-gulose, and D-iditol modified the substrate specificity. Furthermore, when the maltose/glucose of MpGDH in the absence of a substrate specificity modifier is considered as 100%, the value in the presence of D-glucal became 69%, and the substrate specificity was modified.

TABLE 1

| | Wild type MpGDH | | | | |
|---|---|---|---|---|---|
| | | 5 mM | 5 mM | 5 mM | 5 mM |
| Additive | — | Sorbitol | Ribitol | L-gulose | D-iditol |
| Maltose/Glucose (%) | 0.46 | 0.2 | 0.08 | 0.34 | 0.1 |
| Relative value (%) | 100% | 43% | 17% | 74% | 22% |

In the same manner, glucose dehydrogenase (FAD-dependent) (from BBI Solutions, Product Code: GLD1, hereinafter denoted as GLD1) was used to examine the effect of the substrate specificity modifier. Results are shown in Table 2. L-gulose, D-iditol, L-iditol, trehalose, and D-glucal modified the substrate specificity.

TABLE 2

| | | GLD1(Aspergillus) | | | | |
|---|---|---|---|---|---|---|
| | | 5 mM | 5 mM | 5 mM | 5 mM | 5 mM |
| Additive | — | L-gulose | D-iditol | L-iditol | Trehalose | D-glucal |
| Maltose/ | 0.23 | 0 | 0.01 | 0 | 0.08 | 0.09 |
| Glucose (%) | | | | | | |
| Relative | 100% | 0% | 4% | 0% | 35% | 30% |
| value (%) | | | | | | |

In the same manner, MrdGDH was used to examine the effect of the substrate specificity modifier. Results are shown in Table 3. Sorbitol, D-iditol, trehalose, and D-glucal modified the substrate specificity.

TABLE 3

| | MrdGDH | | | | |
|---|---|---|---|---|---|
| | | 5 mM | 5 mM | 5 mM | 5 mM |
| Additive | — | Sorbitol | D-iditol | Trehalose | D-glucal |
| Maltose/Glucose (%) | 23.2 | 17 | 19.3 | 14.4 | 18.1 |
| Relative value (%) | 100% | 73% | 83% | 62% | 78% |

### Example 3

The Effect of a Substrate Specificity Modifier when an Unimmobilized Enzyme is Used

10 μL of 12 mg/ml MpGDH purified preparation, 10 μL of 15 mM each glucose analog, and 10 μL of 20 mM potassium phosphate buffer were mixed and allowed to stand for 2 hours or longer. Onto screen-printed electrodes (manufactured by DropSens, Product number: DRP-C110), 80 μL of PBS, 5 μL of each MpGDH solution, and 10 μL of 2 mg/ml mPMS solution were applied and mixed. Chronoamperometric measurement was carried out at an applied voltage of +100 mV (Ag/Ag+). Once the current value had become sufficiently constant (about 100 seconds after the start of measurement), a maltose solution was added to a final concentration of 5 mM and mixed by pipetting up and down. Measurement was stopped once the current value had become sufficiently constant. The difference in the response current before and after the addition of maltose was regarded as the response current value of maltose. In contrast, when an equivalent amount of pure water was added instead of the maltose solution, increase in the response current was not observed. The used printed electrode was thoroughly washed with ultrapure water and dried, and then chronoamperometric measurement was carried out in the same manner as above. A glucose solution was added instead of the maltose solution and the response current value of glucose was measured. The value calculated by dividing the response current value of maltose by the response current value of glucose was expressed as maltose/glucose (%). When the maltose/glucose of MpGDH is considered as 100%, that in the presence of sorbitol became 67%, that in the presence of ribitol became 58%, that in the presence of L-gulose became 56%, that in the presence of D-iditol became 84%, that in the presence of L-iditol became 68%, and that in the presence of trehalose became 72%, and these substances modified the substrate specificity. L-iditol was found to act as a substrate specificity modifier when the enzyme is not immobilized.

In the same manner, GLD1 was used to examine the effect of the substrate specificity modifier. Results are shown in Table 4. Ribitol, L-gulose, L-iditol, trehalose, and D-glucal modified the substrate specificity.

TABLE 4

| | | GLD1(Aspergillus) | | | | |
|---|---|---|---|---|---|---|
| | | 5 mM | 5 mM | 5 mM | 5 mM | 5 mM |
| Additive | — | Ribitol | L-gulose | L-iditol | Trehalose | D-glucal |
| Maltose/ | 0.33 | 0.29 | 0.23 | 0.25 | 0.28 | 0.25 |
| Glucose (%) | | | | | | |
| Relative | 100% | 88% | 70% | 76% | 85% | 76% |
| value (%) | | | | | | |

In the same manner, MrdGDH was used to examine the effect of the substrate specificity modifier. Results are shown in Table 5. Sorbitol, ribitol, L-gulose, D-iditol, and trehalose modified the substrate specificity.

TABLE 5

| | | MrdGDH | | | | |
|---|---|---|---|---|---|---|
| | | 5 mM | 5 mM | 5 mM | 5 mM | 5 mM |
| Additive | — | Sorbitol | Ribitol | L-gulose | D-iditol | Trehalose |
| Maltose/ | 31 | 23 | 22 | 28 | 21.6 | 22 |
| Glucose (%) | | | | | | |
| Relative | 100% | 74% | 71% | 90% | 70% | 71% |
| value (%) | | | | | | |

### Example 4

The Effect of a Substrate Specificity Modifier when a Carbon Cloth Electrode is Used

80 μl of a multi-walled carbon nanotube solution (1.4 wt %, an MWCNT solution) containing N′N-diphenyl-p-phenylenediamine (DPPD) immobilized by adsorption was applied to a 5 mm by 5 mm square piece of carbon cloth (manufactured by TOYO Corporation) in portions. The cloth was thoroughly dried and then washed with ultrapure water. Subsequently, 40 mg/ml wild type MpGDH and 20 μl of 100 mM various glucose analogs dissolved in ultrapure water were mixed in equal proportions, and 20 μl of the resulting solution was applied to the MWCNT/DPPD immobilized electrode. After being allowed to dry at room temperature, the electrode was placed in 25% glutaraldehyde vapor for 20 minutes to immobilize MpGDH by cross-linking. Then, the electrode was subjected to washing with ultrapure water in the same manner as above to obtain an MWCNT/DPPD/MpGDH-immobilized electrode. Next, this electrode was connected to an ALS electrochemical analyzer 814D. Then, the MWCNT/DPPD/MpGDH-immobilized electrode, an Ag/AgCl reference electrode, and a platinum counter electrode were immersed in PBS, and chronoamperometric measurement was carried out. The applied voltage was set to +250 mV (Ag/AgCl). Once the current value had become sufficiently constant (about 100 seconds after the start of measurement), maltose was added to a final concentration of 1 mM in PBS. Next, subsequently to the addition of the maltose solution, once the current value had become sufficiently constant again (about 50 seconds after the addition of maltose), a glucose solution was added to a final concentration of 1 mM. The difference in the response current before and after the addition of maltose was recorded as the response current value of maltose, and the difference in the response current before and after the addition of glucose was recorded as the response current value of glucose. The value calculated by dividing the response current value of maltose by the response current value of glucose was expressed as maltose/glucose (%). The results are shown in the table below. L-gulose, L-iditol, D-mannitol, glycerol, and xylitol modified the substrate specificity of GDH. The results also indicated that the various glucose analogs were able to modify the substrate specificity of GDH regardless of the form of the electrode.

TABLE 6

| | | Wild type MpGDH | | | | |
|---|---|---|---|---|---|---|
| | | 50 mM | 50 mM | 50 mM | 50 mM | 50 mM |
| Additive | — | L-gulose | L-iditol | D-mannitol | Glycerol | Xylitol |
| Maltose/ | 100% | 62% | 58% | 3% | 29% | 21% |
| Glucose | | | | | | |
| Relative | | | | | | |
| value (%) | | | | | | |

### Example 5

The Effect of a Combination of Substrate Specificity Modifiers when a Carbon Cloth Electrode is Used

An MWCNT/DPPD/MpGDH-immobilized electrode was made in the same manner as Example 4 except that the application step was altered. That is, 20 μl of 40 mg/ml wild type MpGDH was mixed with 10 μl each of 100 mM glycerol and 100 mM L-iditol dissolved in ultrapure water, and 20 μl of the resulting solution was applied to the MWCNT/DPPD immobilized electrode for the application step. Then, chronoamperometric measurement was carried out according to the method described in Example 4, and the maltose/glucose (%) was calculated. As a result, when the maltose/glucose response current value of the MpGDH in the absence of a substrate specificity modifier is considered as 100%, this value in the presence of both glycerol and L-iditol became 8%, and the substrate specificity was modified. Accordingly, the effect of modifying the substrate specificity was greater when both 25 mM L-iditol and glycerol were added compared to when each substance was used alone at 50 mM, demonstrating that the effect of modifying the substrate specificity can be synergistically altered by combining different glucose analogs.

### Example 6

### Constructing a Fuel Cell

The carbon cloth electrode made in Example 4 that used a solution containing a purified GDH and D-mannitol was used as an anode electrode. However, in this Example IPPD was used as the mediator instead of DPPD. Furthermore, a cathode electrode was made by immobilizing 10 mg/ml bilirubin oxidase (BOD, manufactured by Sigma-Aldrich Corporation) by adsorption on carbon cloth onto which a multi-walled carbon nanotube solution (1.4 wt %, an MWCNT solution) was adsorbed. A variable resistance was connected between the wires connecting the anode electrode and the cathode electrode, and additionally an ALS electrochemical analyzer 814D was connected thereto. An electrolyte solution containing PBS (pH 7.4) and 200 mM D-glucose was used as a fuel tank, and the above described anode electrode and cathode electrode were immersed in the tank. Measurement was carried out at room temperature using open circuit potential, which is a potentiostat technique. As a result, a current value of 0.125 mA/cm² was obtained when a resistance of 10 kΩ was connected.

Without wishing to be bound by any specific theory, the fuel cell of the present disclosure can be discussed as follows. That is, as describe in Example 4, using a GDH-immobilized electrode in the presence of various glucose analogs enabled the GDH to react with glucose with a high substrate specificity. Accordingly, for example, a fuel cell using the anode electrode of the present disclosure is expected to enable a certain amount of electrical current to flow when the fuel is a biological component where maltose and glucose coexist, for example even when a maltose concentration temporarily increased due to intake of food, an infusion, and the like. Another possible approach to modification of substrate specificity is modification of enzymes through genetic engineering, which, however, is time-consuming and complicated. According to the present disclosure, the substrate specificity of a GDH can be modified depending on the use. This is beneficial for various uses.

### INDUSTRIAL APPLICABILITY

According to the present disclosure the substrate specificity of glucose dehydrogenase can be modified. This can reduce the effect of maltose when the glucose concentration of a sample is measured using glucose dehydrogenase, enabling more accurate measurement of the glucose concentration. This may be useful for blood glucose control for diabetic patients. Furthermore, by modifying the substrate specificity of glucose dehydrogenase so as to enhance the reactivity to maltose, a higher current value can be obtained when a fuel cell comprising a mixture of glucose and maltose in the fuel thereof is used.

All the references cited in the present specification are incorporated herein by reference in their entirety.

### BRIEF DESCRIPTION OF SEQUENCES

SEQ ID NO: 1 GDH from Mucor prainii (MpGDH) aa
SEQ ID NO: 2 MpGDH gene DNA
SEQ ID NO: 3 Mucor RD056860 GDH (MrdGDH) aa
SEQ ID NO: 4 MrdGDH gene DNA

1. A method of modifying the substrate specificity of a glucose sensor comprising a flavin-adenine-dinucleotide (FAD) dependent glucose dehydrogenase, said method comprising adding a glucose dehydrogenase substrate specificity modifier to the sensor, said modifier being a glucose analog and a low molecular weight compound, wherein the method further comprises bringing the sensor into contact with a sample containing glucose, and a measuring glucose using the sensor comprising the FAD-dependent glucose dehydrogenase (FAD-GDH),
wherein the ratio (Mal/Glu) of the reactivity of the glucose dehydrogenase to maltose (Mal) relative to the reactivity thereof to glucose (Glu) in the presence of the modifier is altered, compared to the ratio (Mal/Glu) of the reactivity of the glucose dehydrogenase to maltose (Mal) relative to the reactivity thereof to glucose (Glu) in the absence of the modifier.

2. The method according to claim 1, wherein the glucose dehydrogenase substrate specificity modifier comprises one or more compounds selected from the group consisting of L-iditol, sorbitol, D-iditol, D-glucal, ribitol, L-gulose, D-mannitol, and glycerol.

3. The method according to claim 1, wherein the glucose dehydrogenase is a glucose dehydrogenase from a microorganism of the genus Mucor or the genus Aspergillus.

4. The method according to claim 1, wherein the glucose dehydrogenase is immobilized onto a solid-phase surface.

5. The method according to claim 1, wherein the glucose dehydrogenase is not immobilized onto a solid-phase surface.

6. A glucose dehydrogenase substrate specificity modifier, said modifier being a glucose analog and a low molecular weight compound, wherein the ratio (Mal/Glu) of the reactivity of an FAD-dependent glucose dehydrogenase (FAD-GDH) to maltose (Mal) relative to the reactivity thereof to glucose (Glu) in the presence of the modifier is altered, compared to the ratio (Mal/Glu) of the reactivity of the glucose dehydrogenase to maltose (Mal) relative to the reactivity thereof to glucose (Glu) in the absence of the modifier.

7. (canceled)

8. The modifier according to claim 6, wherein the glucose dehydrogenase substrate specificity modifier comprises one or more compounds selected from the group consisting of L-iditol, sorbitol, D-iditol, D-glucal, ribitol, L-gulose, D-mannitol, and glycerol.

9. The modifier according to claim 6, wherein the glucose dehydrogenase is a glucose dehydrogenase from a microorganism of the genus Mucor or the genus Aspergillus.

10. A glucose sensor for glucose measurement, comprising the glucose dehydrogenase substrate specificity modifier recited in claim 6 and a flavin-adenine-dinucleotide (FAD) dependent glucose dehydrogenase immobilized onto a solid-phase surface.

11. A glucose sensor for glucose measurement, comprising the glucose dehydrogenase substrate specificity modifier recited in claim 6 and a flavin-adenine-dinucleotide (FAD) dependent glucose dehydrogenase that is not immobilized onto a solid-phase surface.

12. A composition for glucose measurement or a reagent for glucose measurement, comprising the glucose dehydrogenase substrate specificity modifier recited in claim 6 and a flavin-adenine-dinucleotide (FAD) dependent glucose dehydrogenase.

13. The composition for glucose measurement or the reagent for glucose measurement according to claim 12, wherein the composition or the reagent comprises the glucose dehydrogenase substrate specificity modifier and the glucose dehydrogenase as separate reagents.

14. The composition for glucose measurement or the reagent for glucose measurement according to claim 12, wherein the composition or the reagent comprises the glucose dehydrogenase substrate specificity modifier and the glucose dehydrogenase in a single reagent.

15. A glucose measurement method, comprising bringing the glucose sensor of claim 1 into contact with a sample containing glucose, and measuring the glucose in the sample.

16. A screening method for a glucose dehydrogenase substrate specificity modifier, comprising the steps of:
i) providing a glucose dehydrogenase;
ii) determining the ratio (Mal/Glu) of the reactivity of the glucose dehydrogenase to maltose (Mal) relative to the reactivity thereof to glucose (Glu);
iii) contacting the glucose dehydrogenase described above in i) with a candidate substance that is a glucose analog and a low molecular weight compound, and then determining the ratio (Mal/Glu) of the reactivity of the glucose dehydrogenase to maltose (Mal) relative to the reactivity thereof to glucose (Glu) in the presence of the candidate substance;
iv) comparing the ratio (Mal/Glu) obtained in ii) with the ratio (Mal/Glu) obtained in iii) in the presence of the candidate substance; and
v) identifying the candidate substance as a glucose dehydrogenase substrate specificity modifier if the ratio (Mal/Glu) obtained in iii) in the presence of the candidate substance is altered compared to the ratio (Mal/Glu) obtained in ii).

17. A method for producing a reagent for glucose measurement or a composition for glucose measurement, comprising incorporating a glucose dehydrogenase substrate specificity modifier identified by the method recited in claim 16 into the reagent for glucose measurement or the composition for glucose measurement.

18. (canceled)

19. An electrode comprising the glucose dehydrogenase substrate specificity modifier of claim 8 and an FAD-dependent glucose dehydrogenase (FAD-GDH).

20. An fuel cell comprising the glucose dehydrogenase substrate specificity modifier of claim 8 and an FAD-dependent glucose dehydrogenase (FAD-GDH).


SEQUENCE LISTING (4 sequences)

SEQ ID NO: 1 - protein, 641 aa, Mucor prainii [sequence omitted]

SEQ ID NO: 2 - DNA, 1926 bp, Mucor prainii
atgaagatcacagctgccattatcactgttgccacagcatttgcttcttttgcttctgctcaacaagacacaaattcttcctcaactgatacttatgattatgttatcgttggcggcggtgtagctggtttggctttggctagtcgtatctctgaaaacaaggatgtcactgttgctgttctcgagtccggtcctaatgccaatgatagatttgttgtttatgctcctggcatgtatggccaagctgttggcactgatctctgtcctctcattcctactactcctcaagaaaatatgggcaacagaagtctcacaatcgctactggtagattgctcggtggtggcagtgctattaatggtctcgtttggacccgtggtggcttgaaggattacgatgcttgggaggagctcggtaaccctggatggaacggtgccaacttgttcaagtactttaagaaggtcgaaaacttcactcctcctactcctgcccaaattgaatacggcgctacttatcagaaaagtgctcatggcaagaagggacctattgatgtctctttcacgaactacgagttctctcaatctgctagctggaacgcctcactcgaaacccttgatttcactgcacttcctgatatcttgaacggtactttggccggttactctaccactcccaacattttggaccctgagactgttcaacgtgttgattcctatactggttacattgctccttacactagccgtaacaacctcaatgttttggccaaccataccgtctcccgcattcaatttgctcccaagaatggtagcgaacctctcaaggctaccggtgttgaatggtatcccactggcaacaagaatcaaaagcaaattatcaaggcccgttatgaagttatcatctcatctggtgccattggtagtcctaagcttttggaaatctctggtatcggtaataaggatatcgtctctgctgctggtgtcgagtccttgattgacttgcctggcgttggttccaacatgcaagatcacgttcatgctatcactgtctctactaccaatattactggctatactaccaacagcgtctttgtcaatgaaacccttgcccaagaacaaagagaagaatatgaagccaacaagactggtatctgggctactactcccaacaacctcggttatcctacgcccgaacaactcttcaatggcaccgaattcgtttctggaaaggagtttgctgacaagattcgtaactctactgatgaatgggccaactattatgcttccaccaacgcctccaatgtcgagttattaaagaagcaatatgctattgtcgcctctcgttacgaagagaactacttgtctcctattgaaatcaacttcactcctggttatgagggtagcggtaatgtcgatttgcaaaacaacaagtaccaaactgtcaaccatgtcttgattgctcctttaagtcgtggttatactcacattaactcttctgatgtggaggatcattctgtcattaatccccaatactactctcatcctatggatattgatgtccatatcgcttccactaaacttgctcgcgaaatcatcactgcctctcccggtcttggtgacattaacagtggcgaaatcgaacccggtatgaatattacttctgaagacgaccttagatcttggttgagtaataatgtccgttctgactggcatcctgttggtacttgtgctatgcttcccaaggaattaggtggtgttgtcagccccgctctcatggtttacggcacttccaacttgcgtgttgttgatgcttcgattatgcccctcgaagtctcttctcatttgatgcaacccacctacggtattgctgagaaggctgctgacattattaagaatttctacaagactcaacacaagaaccaaaattag

SEQ ID NO: 3 - protein, 631 aa, Mucor RD056860
MRLSVAILTLTSALASVTSAQQNNTDTYDYVIVGGGVGGLALASRLSEDKNVTVAVLESGPYADDKFVVYAPGMYGQAVGTDLCPLLPTVPQPSMNNRTITIATGRLLGGGSAVNGLVWTRGAMKDFDAWQELGNPGWNGTTMFKYFKKIENFHPPTEEQIQYGATYNKSVHGFNGPIDIAFPVFEFPQSANWNASLAHLNFTRRQDLLDGSLHGYSTTPNTLNPQTARRADAYAGYIQPNVNRTNLAVLANHTVSRIQFEARNGSQPLKAIGVEWYTTGGDKTSKQTIKARREIILSSGAIGSPKLLEVSGIGNKAIVTAAGVQSLIDLPGVGSNMQDHVHAVTVSTTNIDGYTTNSVFTNETLAQEQKDLYYNNKTGIWTTTPNNLGYPSPSQLFTNTTFKSGKEFAAMIRNSTDKYAQYYAANNATNVELLKKQYSIVARRYEENYISPIEINFTPGYGGTGMADLQNKKYQTVNHVLVAPLSRGYTHINSSDIEDPVVIDPQYYSHPLDVDVHVASTQLARSILNAPGLASINSGEVEPGEKVQSDEDVRKWLSDNVRSDWHPVGTCAMLPRKLGGVVDSKLKVYGTANLRIVDASIIPLEISSHLMQPVYAVSERAADIIKSSSKK

SEQ ID NO: 4 - DNA, 1896 bp, Artificial
  synthetic
atgcgtctctctgtggcgatcctcactctcacttcggctctggcttcggttacctcggcccaacaaaacaatactgatacttatgactacgtgatcgtcggaggaggagtgggtggactggctctcgcttcgcgcctctccgaggataagaacgttaccgtggctgtcctggaatcgggcccttatgcggatgacaaattcgtggtctacgccccagggatgtatggtcaggctgtcggaactgacctgtgtcctctgctcccaacggttcctcaaccatctatgaacaatcgaaccatcactattgctacgggacgtctgctcggaggaggttcagctgtgaacggactggtctggacccgtggagctatgaaggatttcgacgcttggcaggagctgggaaacccaggatggaatgggaccactatgttcaagtacttcaagaaaatcgaaaacttccatcccccgaccgaggaacagattcaatacggcgctacttataacaagtctgtccacggtttcaatggcccgatcgatattgcctttcccgtgttcgagtttccgcagtctgctaactggaatgcgtcactggcccatctcaacttcacccgccggcaagatctgctcgacggtagtctccacggctacagcacgacccctaacaccctgaatccacagactgcccgacgtgcggatgcctacgctggatatatccaacctaacgtcaatcgaacgaacctggctgtcctcgcgaatcataccgttagtcgcatccagtttgaggcgcggaacggtagccaaccactgaaggccattggcgtggaatggtatactacgggcggagacaagactagtaaacagacgatcaaggcgcgccgggagatcattctgagtagcggagccattgggtcgcctaagctgctcgaagtgtccgggatcggtaacaaagccattgttaccgccgctggagtgcagtctctgatcgatctcccaggcgttggatcaaacatgcaagaccatgtgcacgctgttaccgtgtcgaccactaatatcgatgggtacacgaccaactccgtgttcacgaatgagaccctcgcccaggaacaaaaggacctgtactacaacaacaagactggaatctggactacgacccctaacaatctcgggtatcccagtccgagccagctgttcaccaacactacgtttaagtctggcaaagagtttgcggccatgatccgcaacagtactgataagtacgcccagtactatgctgcgaacaatgctacgaacgtcgagctgctcaagaaacaatatagtatcgtggcccgacgttacgaggaaaactacatcagccctatcgaaatcaacttcacgccaggatacgggggtaccgggatggctgatctgcagaacaagaaatatcaaaccgtgaatcatgtcctggttgcccccctcagtcggggctacactcacatcaactcgtccgatattgaggaccccgttgtgatcgacccgcagtactatagccatccgctggatgtggacgtccacgttgcgagtacccaactggcccgaagcatcctcaacgcccccggactggcttctattaattcaggcgaggtggaaccgggcgagaaggtccagagcgatgaagacgttcgcaaatggctgtcggataacgtgcgttccgactggcatccagtcggaacctgcgctatgctgccacgaaagctcggaggagtcgttgattcgaagctcaaagtctacggcaccgcgaatctgcgtatcgttgacgcctccatcattccgctcgagatttcttcacacctgatgcaaccagtctatgcggtctccgaacgggctgccgacatcatcaaatcctcctctaaaaaataa